(19) United States
(12) Patent Application Publication
(10) Pub. No.: US 2011/0130376 A1
(43) Pub. Date: Jun. 2, 2011

(54) NEW NO RELEASING STEROIDS DERIVATIVES

(75) Inventors: Francesca Benedini, San Donato Milanese (Milano) (IT); Laura Carzaniga, Roncello (MB) (IT); Ennio Ongini, Segrate (Milano) (IT); Stefamo Biondi, Pero (Milano) (IT)

(73) Assignee: NICOX S.A., Sophia Antipolis - Valbonne (FR)

(21) Appl. No.: 13/057,436

(22) PCT Filed: Jul. 24, 2009
(86) PCT No.: PCT/EP2009/059544

§ 371 Date: Feb. 3, 2011

Related U.S. Application Data
(60) Provisional application No. 61/086,305, filed on Aug. 5, 2008.

(51) Int. Cl. A61K 31/573; C07J 41/00; C07J 5/00; A61P 29/00; A61P 25/00; A61P 35/00; A61P 13/12; A61P 11/00; A61P 17/00; A61P 27/02

(52) U.S. Cl. 514/180; 552/575; 552/573; 514/179

(57) ABSTRACT

The invention relates to compounds of general formula (I) and pharmaceutically acceptable salts or stereoisomers thereof

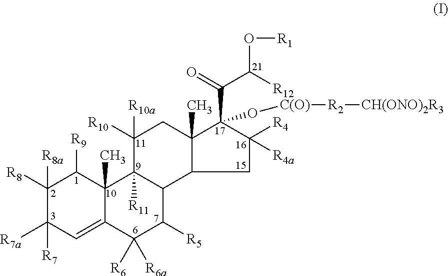

The compounds are useful in the treatment of illnesses wherein the known steroid, parent or precursor steroid, is generally applied, with increased benefit in terms of pharmacological profile and fewer or milder side effects than those of the known steroids.

The present invention relates to nitrooxy derivatives of known steroids, methods for their preparation, pharmaceutical compositions containing these compounds, and methods of using these compounds and compositions for treating illnesses wherein the known steroid, parent or precursor steroid, is generally applied, with increased benefit in terms of pharmacological profile and fewer or milder side effects than those of the known steroids.

Therefore the compounds of the present invention may be used, according to the activity of the parent drug, as drugs having antiinflammatory activity at peripheral level, immunodepressive activity, angiostatic/angiogenetic activity, antiarthritic activity, in the therapy of neurodegenerative diseases on an inflammatory and traumatic basis of the nervous system, in the therapy of respiratory diseases such as asthma and COPD, in substitutive hormonal therapies, preferably in the post-menopause therapy, in rheumatic disease therapies, in renal disease therapies, in ocular disease therapies such as ocular hypertension, age-related macular degeneration, diabetic macular edema, diabetic retinopathy, hypertensive retinopathy and retinal vasculopathies, in dermatological disease therapies, in autoimmune disease therapy in tumoral process therapies, in inflammatory pathologies affecting the gastrointestinal system.

In the prior art nitrooxy derivatives of steroids, which are usable also as cardiovascular agents for the coronary insufficiency or angina pectoris therapy, are described.

For example, German patent DE 2,222,491 describes the preparation of pregnane derivatives having in position 21 the —CH₂—O—NO₂ group. In said patent it is stated that said derivatives have a cardiotropic activity. This activity represents a drawback for said compounds, since they modify the cardiac frequency.

U.S. Pat. No. 3,494,941 describes steroid derivatives from 3-hydroxy-extrane or from extr-4 en-3 one, used as vasodilators in the treatment of cardiac affections such as coronary insufficiency and angina pectoris. In the structure of said compounds a ONO₂ group is at the free end of the alkylene chain which is linked by an ether bond to the steroid in position 17. According to said patent it is possible to have nitrate groups also in the positions 3 and 16 of the steroidal structure. The same drawbacks mentioned above as regards the effects on the cardiac frequency can be repeated for the compounds of this patent.

U.S. Pat. No. 3,183,252 describes derivatives of 16-nitrate-alkylpregnanes wherein the alkyl group is linked to the pregnane structure by a carbon-carbon bond. The compounds according to said patent can be used as vasodilators. The same drawbacks reported for the above prior art can be repeated.

WO 98/15568 and WO 03/064443 in the name of the Applicant describe nitrate esters of steroidal compounds, wherein between the steroidal structure and the nitrooxy group a bivalent linking group is inserted. Said compounds show a good efficacy and/or good tolerability with respect to the corresponding precursors.

Patent application WO 00/61604 in the name of the Applicant describes nitrooxy derivatives of steroidal compounds with various linking groups having at one end a nitrooxy group, and covalently linked with the other end to a steroidal compound. In said application the uses concern the compounds usable in the treatment of patients in oxidative stress. Said compounds contain in the molecule also a bivalent linking group which must be capable to prevent the free radicals production and is selected on the basis of the tests reported therein.

EP 1 336 602 describes new pharmacological compounds which can release nitric oxide and their use for the prevention and treatment of inflammatory, ischemic, degenerative and proliferative diseases. These compounds have a slower absorption compared to classic nitrate vasodilators. Between the compounds, steroidal nitroderivatives are disclosed.

WO 00/499993 describes nitrite, nitrate, thionitrite or thionitrate steroid derivatives optionally substituted in position 3, 11, 17 or 21 with a nitrate ester.

The Applicant has surprisingly and unexpectedly found a class of nitric oxide releasing compounds with a better bioavailability and/or a prolonged release of NO in comparison with the compounds known in prior art. In general the compounds of the present invention have a better drugability in comparison to the corresponding compounds of the prior art.

An object of the present invention is a compound of general formula (I) and pharmaceutically acceptable salts or stereoisomers thereof

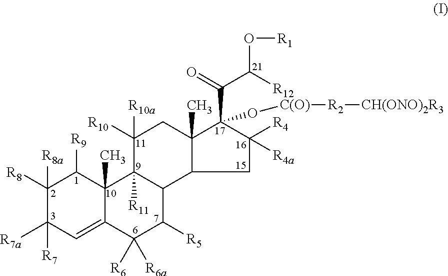

wherein:
R₁ is —H or R₁ is selected from
   (A) —R¹—CH(NHR²)—C(O)—O—Y
   (B) —R¹—CH(COOH)NH—C(O)—Y
   (C) —R¹—CH(COOH)—O—C(O)—Y
   (D) —C(O)CH(R³)—NH—C(O)—Y
   (E) —C(O)CH₂—CH(R⁴)—NH—C(O)—Y
   (F) —(Z)—Y
   (G)

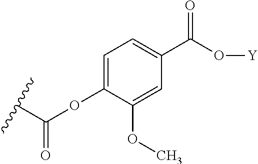

(H)

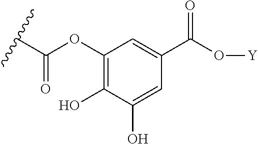

(I)

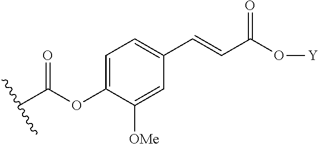

wherein:
R¹ is selected from
   R^{1a})

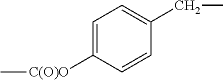

—C(O)—S—CH₂—, —C(O)O—CH(CH₃)—, —C(O)O—CH₂—;
preferably R^{1a} is

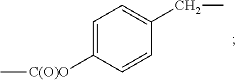

R^{1b})
—C(O)—CH₂—, —C(O)—(CH₂)₂—; preferably R^{1b} is —C(O)—CH₂—;
R² is —H or —C(O)CH₃;
R³ is —H, —CH₃, isopropyl, isobutyl, sec-butyl, methylthio-(CH₂)₂—, benzyl, preferably R³ is H or —CH₃;
R⁴ is —H, —CH₃, isopropyl, isobutyl, sec-butyl, methylthio-(CH₂)₂—, benzyl, preferably R⁴ is H or —CH₃;
Z is —C(O) or —C(O)—X″, wherein X″ is O, S or NR₁₁ wherein R₁₁ is H or a C₁-C₄ alkyl; preferably X″ is O;
R₂ is a straight or branched C₁₀ alkylene; preferably R₂ is a straight C₁-C₆ alkylene;
R₃ is H or a straight or branched C₁-C₄ alkyl, preferably R₃ is H or —CH₃, more preferably R₃ is H;
R₄ is —H, —CH₃;
R_{4A} is —H,
or R₄ and R_{4A} taken together are ═CH₂;
R₅ is —H, Cl;
R₆ is —H, Cl, F, CH₃;
R₆ₐ is —H,
or R₆ and R₅ taken together are a double bond;
R₇ₐ is H,
or R₇ and R_{7A} taken together are a ═O;
R₈ is H, Cl,
or R₇ and R₈ taken together are the group of formula (V)

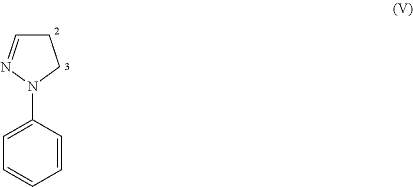

R₈ₐ is H,
R₉ is —H,
or R₈ₐ and R₉ taken together are a double bond
R₁₀ is —OH,
R₁₀ₐ is H,
or R₁₀ and R₁₀ₐ taken together are ═O;
R₁₁ is —H, —Cl, —F;
R₁₂ is —H, CH₃;
   wherein R₄, R₄ₐ, R₅, R₆, R₆ₐ, R₇, R₇ₐ, R₈, R₈ₐ, R₉, R₁₀, R₁₀ₐ can be linked to the corresponding carbon atoms of the steroidal structure in position α or β;
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
   wherein
R₁₃ is a straight or branched C₁-C₁₀ alkylene; preferably R₁₃ is a straight C₁-C₈ alkylene;
R₁₄ is H or a straight or branched C₁-C₄ alkyl, preferably R₁₄ is H or —CH₃;
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl; preferably X is O;
preferably Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O;
excluding the following structures from formula (I):

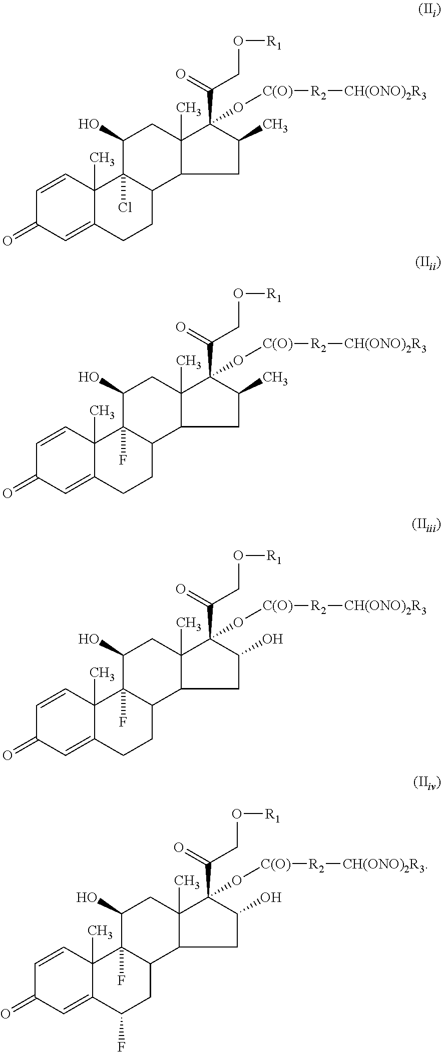

Another embodiment of the present invention relates to compounds of formula (I) wherein
R₁ is selected from
   (A) —R¹—CH(NHR²)—C(O)—O—Y
   (B) —R¹—CH(COOH)NH—C(O)—Y
   (C) —R¹—CH(COOH)—O—C(O)—Y
   (D) —C(O)CH(R³)—NH—C(O)—Y
   (E) —C(O)CH₂—CH(R⁴)—NH—C(O)—Y
   (F) —(Z)—Y
   (G)

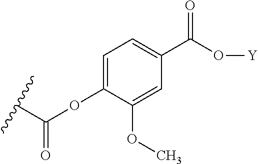

(H)

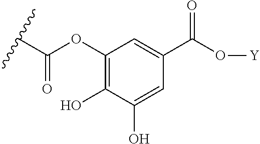

(I)

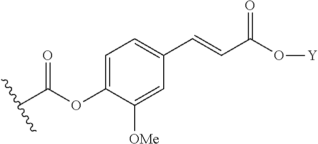

wherein:
R¹ is selected from
   R^{1a})

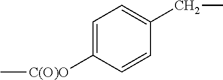

—C(O)—S—CH₂—, —C(O)O—CH(CH₃)—, —C(O)O—CH₂—;
preferably R^{1a} is

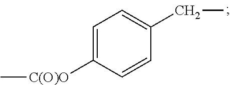

R^{1b})
—C(O)—CH₂—, —C(O)—(CH₂)₂—; preferably R^{1b} is —C(O)—CH₂—;
R² is —H or —C(O)CH₃;
R³ is —H, —CH₃, isopropyl, isobutyl, sec-butyl, methylthio-(CH₂)₂—, benzyl, preferably R³ is H;
R⁴ is —H, —CH₃, isopropyl, isobutyl, sec-butyl, methylthio-(CH₂)₂—, benzyl, preferably R⁴ is H;
Z is —C(O) or —C(O)—X″, wherein X″ is O, S or NR₁₁ wherein R₁₁ is H or a C₁-C₄ alkyl; preferably X″ is O;
R₂ is a straight or branched C₁-C₁₀ alkylene; preferably R₂ is a straight C₁-C₅ alkylene;
R₃ is H or a straight or branched C₁-C₄ alkyl, preferably R₃ is H or —CH₃;
R₄ is —CH₃, and it is linked to the 16 position of the steroidal structure in position β;
R_{4A} is —H;
R₅ is —H;
R₆ is —H;
R₆ₐ is —H;
R₇ and R_{7A} taken together are a ═O;
R₈ is H;
R₈ₐ and R₉ taken together are a double bond;
R₁₀ is —OH, and it is linked to the corresponding carbon atoms of the steroidal structure in position u;
R₁₀ₐ is H;
R₁₁ is —F;
R₁₂ is —H, CH₃;
Y is selected from
—R₁₃—CH(ONO₂)R₁₄
—R₁₃—CH(ONO₂)—(CR₁₅R₁₆)ₜ—CH(ONO₂)R₁₄
[(CH₂)ₒ—X]ₚ—[(CH₂)ᵣ—X]ₛ—(CH₂)_{q}—(CR₁₅R₁₆)ₜ—CH(ONO₂)R₁₄
[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
   wherein
R₁₃ is a straight or branched C₁-C₁₀ alkylene; preferably R₁₃ is a straight C₁-C₆ alkylene;
R₁₄ is H or a straight or branched C₁-C₄ alkyl, preferably R₁₄ is H or —CH₃;
R¹⁵ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl; preferably X is O;
preferably Y is selected from
—R₁₃—CH(ONO₂)R₁₄
—R₁₃—CH(ONO₂)—(CR₁₅R₁₆)ₜ—CH(ONO₂)R₁₄
[(CH₂)ₒ—X]ₚ—[(CH₂)ᵣ—X]ₛ—(CH₂)_{q}—(CR₁₅R₁₆)ₜ—CH(ONO₂)R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O.

Another embodiment of the present invention relates to compounds of formula (I) wherein
R₁ is selected from
   (A) —R¹—CH(NHR²)—C(O)—O—Y
   (B) —R¹—CH(COOH)NH—C(O)—Y
   (C) —R¹—CH(COOH)—O—C(O)—Y
   (D) —C(O)CH(R³)—NH—C(O)—Y
   (E) —C(O)CH₂—CH(R⁴)—NH—C(O)—Y
   (F) —(Z)—Y
   (G)

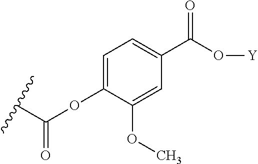

(H)

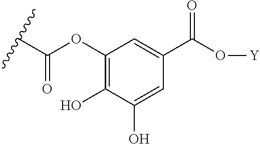

(I)

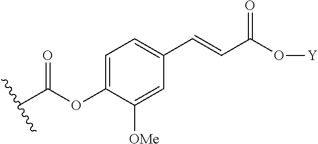

wherein:
R¹ is selected from
   R^{1a})

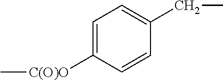

—C(O)—S—CH₂—, —C(O)O—CH(CH₃)—, —C(O)O—CH₂—;
preferably R^{1a} is

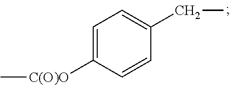

R^{1b})
—C(O)—CH₂—, —C(O)—(CH₂)₂—; preferably R^{1b} is —C(O)—CH₂—;
R² is —H or —C(O)CH₃;
R³ is —H, —CH₃, isopropyl, isobutyl, sec-butyl, methylthio-(CH₂)₂—, benzyl, preferably R³ is H;
R⁴ is —H, —CH₃, isopropyl, isobutyl, sec-butyl, methylthio-(CH₂)₂—, benzyl, preferably R⁴ is H;
Z is —C(O) or —C(O)—X″, wherein X″ is O, S or NR₁₁ wherein R₁₁ is H or a C₁-C₄ alkyl; preferably X″ is O;
R₂ is a straight or branched C₁-C₁₀ alkylene; preferably R₂ is a straight C₁-C₈ alkylene;
R₃ is H or a straight or branched C₁-C₄ alkyl, preferably R₃ is H or —CH₃;
R₄ is —CH₃, and it is linked to the corresponding carbon atoms of the steroidal structure in position β;
R_{4A} is —H;
R₅ is —H;
R₆ is —F and it is linked to the corresponding carbon atoms of the steroidal structure in position β;
R₆ₐ is —H;

R₇ and R_{7A} taken together are a ═O;
R₈ is H;
R₈ₐ and R₉ taken together are a double bond;
R₁₀ is —OH, and it is linked to the corresponding carbon atoms of the steroidal structure in position α;
R₁₀ₐ is H;
R₁₁ is —F;
R₁₂ is —H, CH₃;
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
   wherein
R₁₃ is a straight or branched C₁-C₁₀ alkylene; preferably
R₁₃ is a straight C₁-C₆ alkylene;
R₁₄ is H or a straight or branched C₁-C₄ alkyl, preferably R₁₄ is H or —CH₃;
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl;
preferably X is O;
preferably Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O.

Another embodiment of the present invention relates to compounds of formula (I) wherein
R₁ is selected from
   (A) —R¹—CH(NHR²)—C(O)—O—Y
   (B) —R¹—CH(COOH)NH—C(O)—Y
   (C)—R¹—CH(COOH)—O—C(O)—Y
   (D) —C(O)CH(R³)—NH—C(O)—Y
   (E) —C(O)CH₂—CH(R⁴)—NH—C(O)—Y
   (F) —(Z)—Y
   (G)

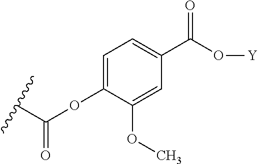

(H)

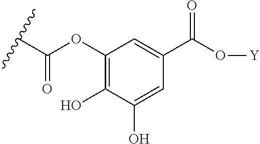

(I)

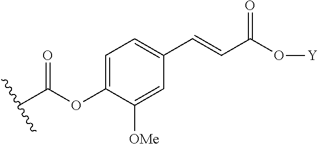

wherein:
R¹ is selected from
   R^{1a})

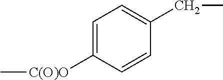

—C(O)—S—CH₂—, —C(O)O—CH(CH₃)—, —C(O)O—CH₂—;
preferably R^{1a} is

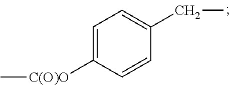

R^{1b})
—C(O)—CH₂—, —C(O)—(CH₂)₂—; preferably R^{1b} is —C(O)—CH₂—;
R² is —H or —C(O)CH₃;
R³ is —H, —CH₃, isopropyl, isobutyl, sec-butyl, methylthio-(CH₂)₂—, benzyl, preferably R³ is H;
R⁴ is —H, —CH₃, isopropyl, isobutyl, sec-butyl, methylthio-(CH₂)₂—, benzyl, preferably R⁴ is H;
Z is —C(O) or —C(O)—X″, wherein X″ is O, S or NR₁₁ wherein R₁₁ is H or a C₁-C₄ alkyl; preferably X″ is O;
R₂ is a straight or branched C₁-C₁₀ alkylene; preferably R₂ is a straight C₁-C₆ alkylene;
R₃ is H or a straight or branched C₁-C₄ alkyl, preferably R₃ is H or
R₄ is —H;
R_{4A} is —H;
R₅ is —H;
R₆ is —H;
R₆ₐ is —H,
R₇ and R_{7A} taken together are a ═O;
R₈ is H;
R₈ₐ is H;
R₉ is —H;
R₁₀ is —OH, and it is linked to the corresponding carbon atoms of the steroidal structure in position α;
R₁₀ₐ is H;
R₁₁ is H;
R₁₂ is —H, CH₃;
Y is selected from
—R₁₃—CH(ONO₂)R₁₄
—R₁₃—CH(ONO₂)—(CR₁₅R₁₆)ₜ—CH(ONO₂)R₁₄
[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CH₁₅R₁₆)ₜ—CH(ONO₂)R₁₄
   wherein
R₁₃ is a straight or branched C₁-C₁₀ alkylene; preferably R₁₃ is a straight C₁-C₆ alkylene;
R₁₄ is H or a straight or branched C₁-C₄ alkyl, preferably R₁₄ is H or —CH₃;
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl; preferably X is O;
preferably Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂) R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O.

Another embodiment of the present invention relates to compounds of formula (I) wherein
R₁ is selected from
   (A) —R¹—CH(NHR²)—C(O)—O—Y
   (B) —R¹—CH(COOH)NH—C(O)—Y
   (C)—R¹—CH(COOH)—O—C(O)—Y
   (D) —C(O)CH(R³)—NH—C(O)—Y
   (E) —C(O)CH₂—CH(R⁴)—NH—C(O)—Y
   (F) —(Z)—Y
   (G)

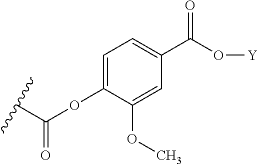

(H)

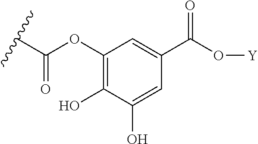

(I)

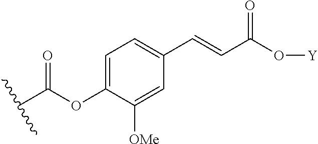

wherein:
R¹ is selected from
   R^{1a})

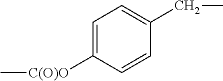

—C(O)—S—CH₂—, —C(O)O—CH(CH₃)—, —C(O)O—CH₂—;
preferably R^{1a} is

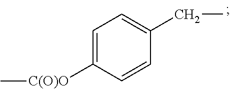

R^{1b})
—C(O)—CH₂—, —C(O)—(CH₂)₂—; preferably R^{1b} is —C(O)—CH₂—;
R² is —H or —C(O)CH₃;
R³ is —H, —CH₃, isopropyl, isobutyl, sec-butyl, methylthio-(CH₂)₂—, benzyl, preferably R³ is H;
R⁴ is —H, —CH₃, isopropyl, isobutyl, sec-butyl, methylthio-(CH₂)₂—, benzyl, preferably R⁴ is H;
Z is —C(O) or —C(O)—X″, wherein X″ is O, S or NR₁₁ wherein R₂₂ is H or a C₁-C₄ alkyl; preferably X″ is O;
R₂ is a straight or branched C₁-C₁₀ alkylene; preferably R₂ is a straight C₂-C₅ alkylene;
R₃ is H or a straight or branched C₁-C₄ alkyl, preferably R₃ is H or —CH₃;
R₄ is —H;
R_{4A} is —H;
R₅ is —H;
R₆ is —H;
R₆ₐ is —H,
R₇ and R_{7A} taken together are a ═O;
R₈ is H;
R₈ₐ is H;
R₉ is —H;
R₁₀ is —OH, and it is linked to the corresponding carbon atoms of the steroidal structure in position α;
R₁₀ₐ is H;
R₁₁ is H;
R₁₂ is —H, CH₃;
Y is selected from
—R₁₃—CH(ONO₂)R₁₄
—R₁₃—CH(ONO₂)—(CR₁₅R₁₆)ₜ—CH(ONO₂)R₁₄
[(CH₂)ₒ—X]ₚ—[(CH₂)ᵣ—X]ₛ—(CH₂)_{q}—(CR₁₅R₁₆)ₜ—CH(ONO₂)R₁₄
[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
   wherein
R₁₃ is a straight or branched C₁-C₁₀ alkylene; preferably R₁₃ is a straight C₁-C₆ alkylene;
R₁₄ is H or a straight or branched C₁-C₄ alkyl, preferably R₁₄ is H or
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl;
preferably X is O;
preferably Y is selected from —R₁₃—CH(ONO₂)R₁₄
—R₁₃—CH(ONO₂)—(CR₁₅R₁₆)ₜ—CH(ONO₂)R₁₄
[(CH₂)ₒ—X]ₚ—[(CH₂)ᵣ—X]ₛ—(CH₂)_{q}—(CR₁₅R₁₆)ₜ—CH(ONO₂)R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O.

Another embodiment of the present invention relates to compounds of formula (I) wherein
R₁ is —H;
R₂ is a straight or branched C₁-C₁₀ alkylene; preferably R₂ is a straight C₁-C₅ alkylene;
R₃ is H or a straight or branched C₁-C₄ alkyl, preferably R₃ is H or
R₄ is —CH₃, and it is linked to the 16 position of the steroidal structure in position β;
R_{4A} is —H;
R₅ is —H;
R₆ is —H;
R₆ₐ is —H;
R₇ and R_{7A} taken together are a ═O;
R₈ is H;
R₈ₐ and R₉ taken together are a double bond;
R₁₀ is —OH, and it is linked to the corresponding carbon atoms of the steroidal structure in position α;
R₁₀ₐ is H;
R₁₁ is —F;
R₁₂ is —H, CH₃.

Another embodiment of the present invention relates to compounds of formula (I) wherein
R₁ is —H;
R₂ is a straight or branched C₁-C₁₀ alkylene; preferably R₂ is a straight C₁-C₈ alkylene;
R₃ is H or a straight or branched C₁-C₄ alkyl, preferably R₃ is H or —CH₃;
R₄ is —CH₃, and it is linked to the corresponding carbon atoms of the steroidal structure in position β;
R_{4A} is —H;
R₅ is —H;
R₆ is —F and it is linked to the corresponding carbon atoms of the steroidal structure in position β;
R₆ₐ is —H;
R₇ and R_{7A} taken together are a ═O;
R₈ is H;
R₈ₐ and R₉ taken together are a double bond;
R₁₀ is —OH, and it is linked to the corresponding carbon atoms of the steroidal structure in position α;
R₁₀ₐ is H;
R₁₁ is —F;
R₁₂ is —H, CH₃.

Another embodiment of the present invention relates to compounds of formula (I) wherein
R₁ is —H;
R₂ is a straight or branched C₁-C₁₀ alkylene; preferably R₂ is a straight C₁-C₅ alkylene;
R₃ is H or a straight or branched C₁-C₄ alkyl, preferably R₃ is H or —CH₃;
R₄ is —H;
R_{4A} is —H;
R₅ is —H;
R₆ is —H;
R₆ₐ is —H,
R₇ and R_{7A} taken together are a ═O;
R₈ is H;
R₈ₐ is H;
R₉ is —H;
R₁₀ is —OH, and it is linked to the corresponding carbon atoms of the steroidal structure in position α;
R₁₀ₐ is H;
R₁₁ is H;
R₁₂ is —H, CH₃.

Another embodiment of the present invention relates to compounds of formula (I) wherein
R₁ is —H;
R₂ is a straight or branched C₁-C₁₀ alkylene; preferably R₂ is a straight C₁-C₅ alkylene;
R₃ is H or a straight or branched C₁-C₄ alkyl, preferably R₃ is H or —CH₃;
R₄ is —H;
R_{4A} is —H;
R₅ is —H;
R₆ is —H;
R₆ₐ is —H,
R₇ and R_{7A} taken together are a ═O;
R₈ is H;
R₈ₐ is H;
R₉ is —H;
R₁₀ is —OH, and it is linked to the corresponding carbon atoms of the steroidal structure in position α;
R₁₀ₐ is H;
R₁₁ is H;
R₁₂ is —H, CH₃.

Another embodiment of the invention provides a compound of general formula (I) and pharmaceutically acceptable salts or stereoisomers thereof

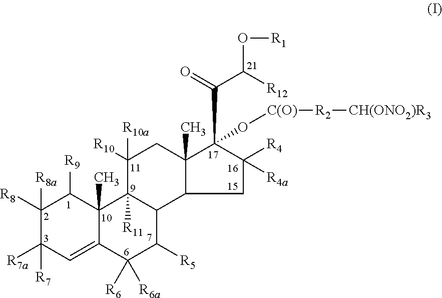

wherein

R₁ is
(F) —(Z)—Y
   wherein:
Z is —C(O) or —C(O)—X″, wherein X″ is O, S or NR₁₁ wherein R₁₁ is H or a C₁-C₄ alkyl; preferably X″ is O;
R₂ is a straight or branched C₁-C₁₀ alkylene; preferably R₂ is a straight C₁-C₆ alkylene;
R₃ is H or a straight or branched C₁-C₄ alkyl, preferably R₃ is H or —CH₃, more preferably R₂ is H;
R₄ is —H, —CH₃;
R_{4A} is —H,
or R₄ and R_{4A} taken together are ═CH₂;
R₅ is —H, Cl;
R₆ is —H, Cl, F, CH₃;
R₆ₐ is —H,
or R₆ and R₅ taken together are a double bond;
R₇ₐ is H,
R₇ and R_{7A} taken together are a ═O;
R₈ is H, Cl,
or R₇ and R₈ taken together are the group of formula (V)

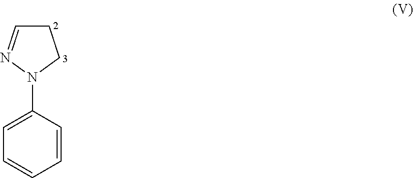

R₈ₐ is H,
R₉ is —H,
or R₈ₐ and R₉ taken together are a double bond
R₁₀ is —OH,
R₁₀ₐ is H,
or R₁₀ and R₁₀ₐ taken together are ═O;
R₁₁ is —H, —F;
R₁₂ is —H, CH₃;
   wherein R₄, R₄ₐ, R₅, R₆, R₆ₐ, R₇, R₇ₐ, R₈, R₈ₐ, R₉, R₁₀, R₁₀ₐ can be linked to the corresponding carbon atoms of the steroidal structure in position α or β;
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
   wherein
R₁₃ is a straight or branched C₁-C₁₀ alkylene; preferably
R₁₃ is a straight C₁-C₆ alkylene;
R₁₄ is H or a straight or branched C₁-C₄ alkyl, preferably R₁₄ is H or —CH₃;
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl;
preferably X is O;
preferably Y is selected from
—R₁₃—OH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O;
excluding the following structures from formula (I):

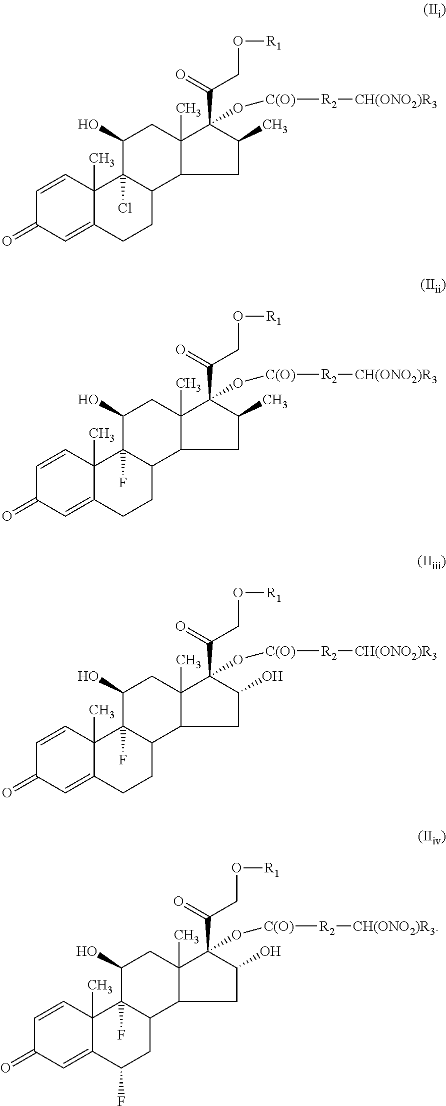

Another embodiment of the invention provides a compound of general formula (I) and pharmaceutically acceptable salts or stereoisomers thereof

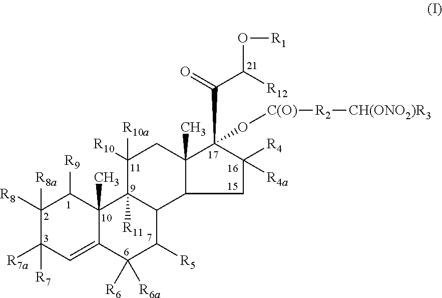

wherein
R₁ is
   (G)

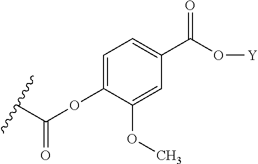

(H)

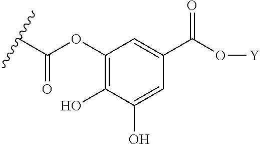

(I)

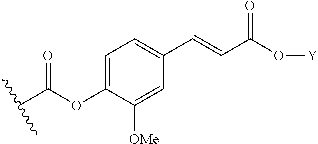

wherein:
R₂ is a straight or branched C₁-C₁₀ alkylene; preferably R₂ is a straight C₁-C₆ alkylene;
R₃ is H or a straight or branched C₁-C₄ alkyl, preferably R₃ is H or —CH₃, more preferably R₃ is H;
R₄ is —H, —CH₃;
R_{4A} is —H,
or R₄ and R_{4A} taken together are ═CH₂;
R₅ is —H, Cl;
R₆ is —H, Cl, F, CH₃;
R₆ₐ is —H,
or R₆ and R₅ taken together are a double bond;
R₇ₐ is H,
R₇ and R_{7A} taken together are a ═O;
R₈ is H, Cl,
or R₇ and R₈ taken together are the group of formula (V)

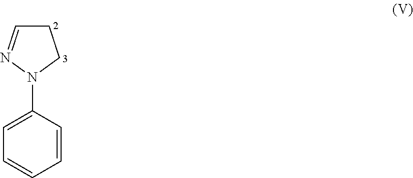

R₈ₐ is H,
R₉ is —H,
or Rₐ and R₉ taken together are a double bond
R₁₀ is —OH,
R₁₀ₐ is H,
or R₁₀ and R₁₀ₐ taken together are ═O;
R₁₁ is —H, —Cl, —F;
R₁₂ is —H, CH₃;
   wherein R₄, R₄ₐ, R₅, R₆, R₆ₐ, R₇, R₇ₐ, R₈, R₈ₐ, R₉, R₁₀, R₁₀ₐ can be linked to the corresponding carbon atoms of the steroidal structure in position α or β;
Y is selected from
—R₁₃—CH(ONO₂)R₁₄
—R₁₃—CH(ONO₂)—(CR₁₅R₁₆)₅—CH(ONO₂)R₁₄
[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CR₁₅R₁₆)ₜCH(ONO₂)
   wherein
R₁₃ is a straight or branched C₁-C₁₀ alkylene; preferably R₁₃ is a straight C₁-C₆ alkylene;
R₁₄ is H or a straight or branched C₁-C₄ alkyl, preferably R₁₄ is H or —CH₃;
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl; preferably X is O;
preferably Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O;
excluding the following structures from formula (I):

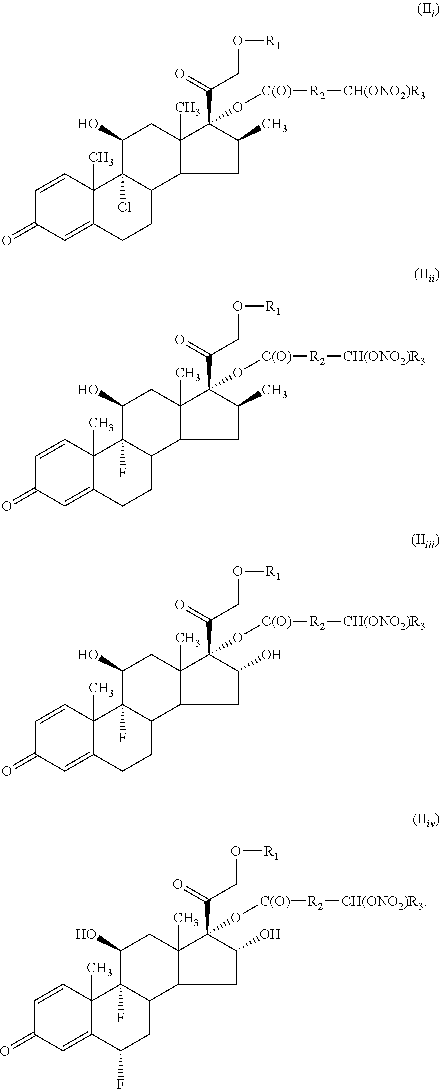

Another embodiment of the invention provides a compound of general formula (I) and pharmaceutically acceptable salts or stereoisomers thereof

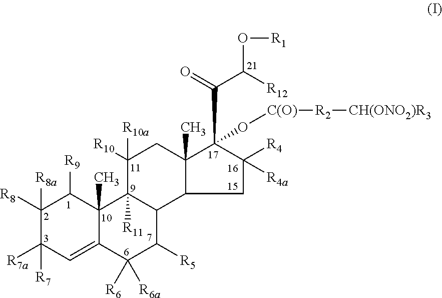

wherein
R₁ is
   (A) —R¹—CH(NHR²)—C(O)—O—Y
   (B) —R¹—CH(COOH)NH—C(O)—Y
   (C) —R¹—CH(COOH)—O—C(O)—Y
   (D) —C(O)CH(R³)—NH—C(O)—Y
   (E) —C(O)CH₂—CH(R⁴)—NH—C(O)—Y
      wherein:
R¹ is selected from:
   R^{1a})

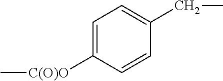

—C(O)—S—CH₂—, —C(O)O—CH(CH₃)—, —C(O)O—CH₂—;
preferably R^{1a} is

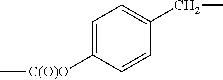

R^{1b})
—C(O)—CH₂—, —C(O)—(CH₂)₂—; preferably R^{1b} is —C(O)—CH₂—;
R² is —H or —C(O)CH₃;
R³ is —H, —CH₃, isopropyl, isobutyl, sec-butyl, methylthio-(CH₂)₂₋, benzyl; preferably R³ is H or —CH₃;
R⁴ is —H, —CH₃, isopropyl, isobutyl, sec-butyl, methylthio-(CH₂)₂—, benzyl; preferably R⁴ is H or —CH₃;
R₂ is a straight or branched C₁-C₁₀ alkylene; preferably R₂ is a straight C₁-C₆ alkylene;
R₃ is H or a straight or branched C₁-C₄ alkyl, preferably R₃ is H or —CH₃, more preferably R₆ is H;
R₄ is —H, —CH₃;
R_{4A} is —H,
or R₄ and R_{4A} taken together are ═CH₂
R₅ is —H, Cl;
R₆ is —H, Cl, F, CH₃;
R₆ₐ is —H,
or R₆ and R₅ taken together are a double bond;
R₇ₐ is H,
R₇ and R_{7A} taken together are a ═O;
R₉ is H, Cl,
or R₇ and R₈ taken together are the group of formula (V)

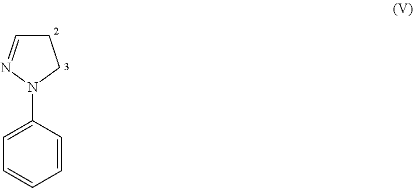

R₈ₐ is H,
R₉ is —H,
or R₈ₐ and R₉ taken together are a double bond
R₁₀ is —OH,
R₁₀ₐ is H,
or R₁₀ and R₁₀ₐ taken together are ═O;
R₁₁ is —H, —Cl, —F;
R₁₂ is —H, CH₃;
   wherein R₄, R₄ₐ, R₅, R₆, R₆ₐ, R₇, R₇ₐ, R₈, R₈ₐ, R₉, R₁₀, R₁₀ₐ can be linked to the corresponding carbon atoms of the steroidal structure in position α or β;
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ₜ—X]ₛ—(CH₂)_{q}—CH(ONO₂)—(CR₁₅R₁₆)ₜ—CH(ONO₂)R₁₄
   wherein
R₁₃ is a straight or branched C₁-C₁₀ alkylene; preferably R₁₃ is a straight C₁-C₆ alkylene;
R₁₄ is H or a straight or branched C₁-C₄ alkyl, preferably R₁₄ is H or —CH₃;
R₁₅ and R₁₆ are at each occurrence independently H or a straight or branched C₁-C₁₀ alkylene, preferably R₁₅ and R₁₆ are H or —CH₃;
o and r are integers from 1 to 6; preferably o and r are integers from 1 to 4, more preferably o is 1 and r is 2;
p and s are integers from 1 to 6; preferably p and s are integers from 1 to 4; more preferably p and s are 1;
q is an integer from 0 to 6; preferably q is from 0 to 4, more preferably q is 0 or 1;
t is an integer from 0 to 6; preferably t is from 0 to 4, more preferably t is 0 or 1;
X is O, S or NR₁₇ wherein R₁₇ is H or a C₁-C₄ alkyl;
preferably X is O;
preferably Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂) R₁₄
   wherein
R₁₃ is a straight C₁-C₆ alkylene;
R₁₄ is H or —CH₃;
R₁₅ and R₁₆ at each occurrence are independently H or —CH₃;
o and r are integers from 1 to 4,
p and s are from 1 to 4;
q is from 0 to 4,
t is 0 or 1,
X is O;
excluding the following structures from formula (I):

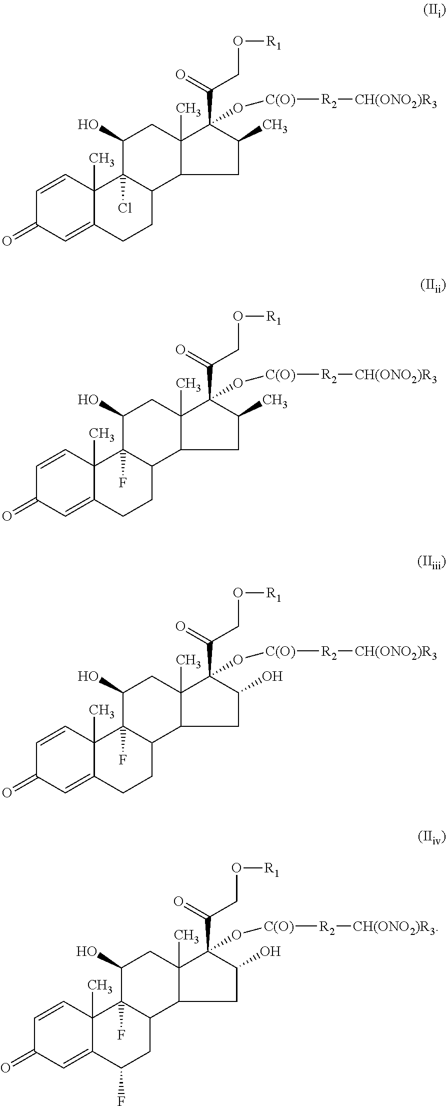

Another embodiment of the invention provides a compound selected from the group:

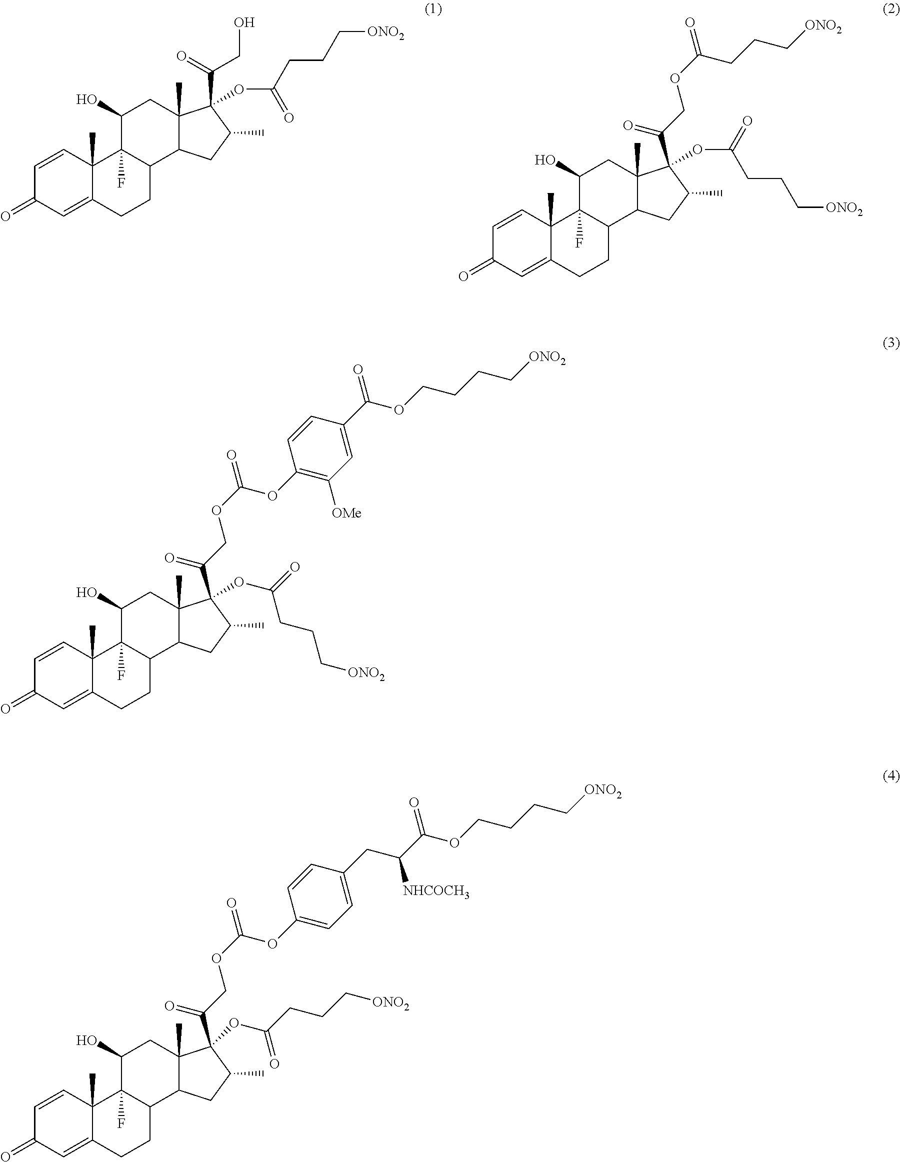

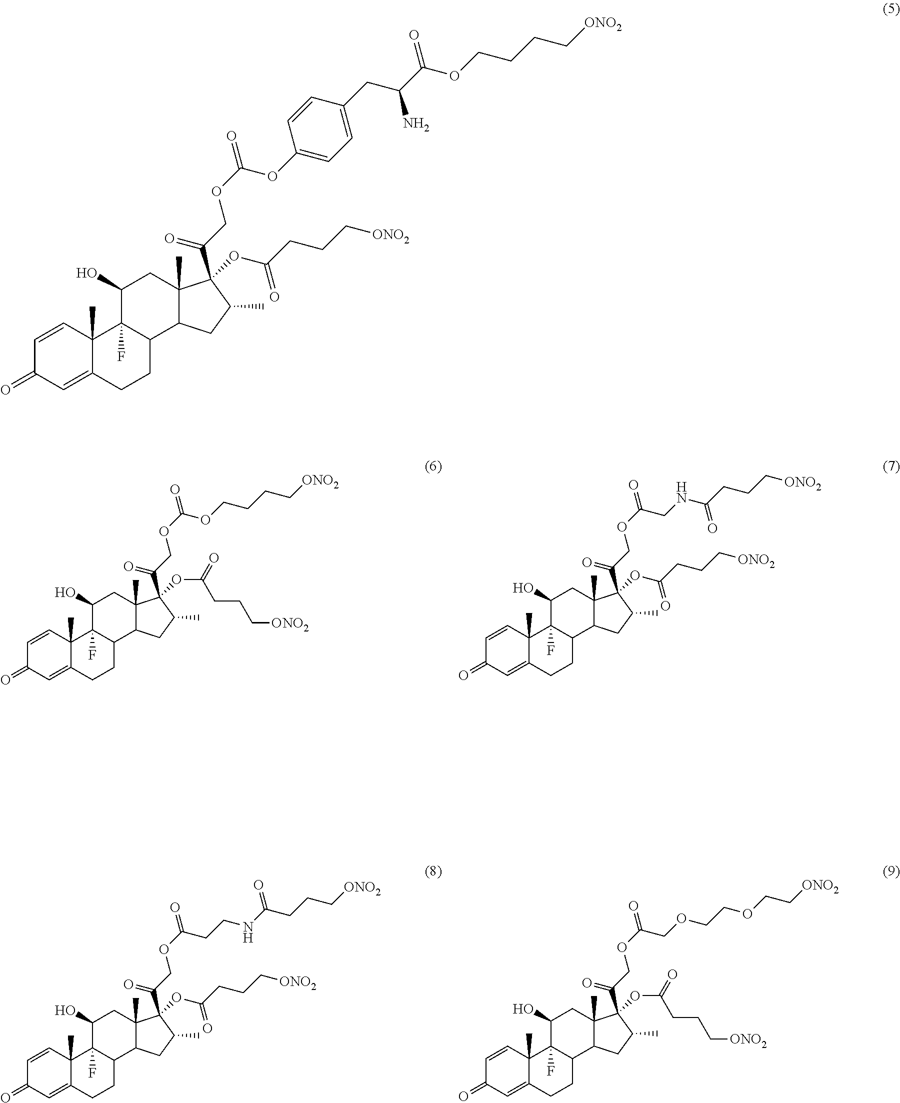

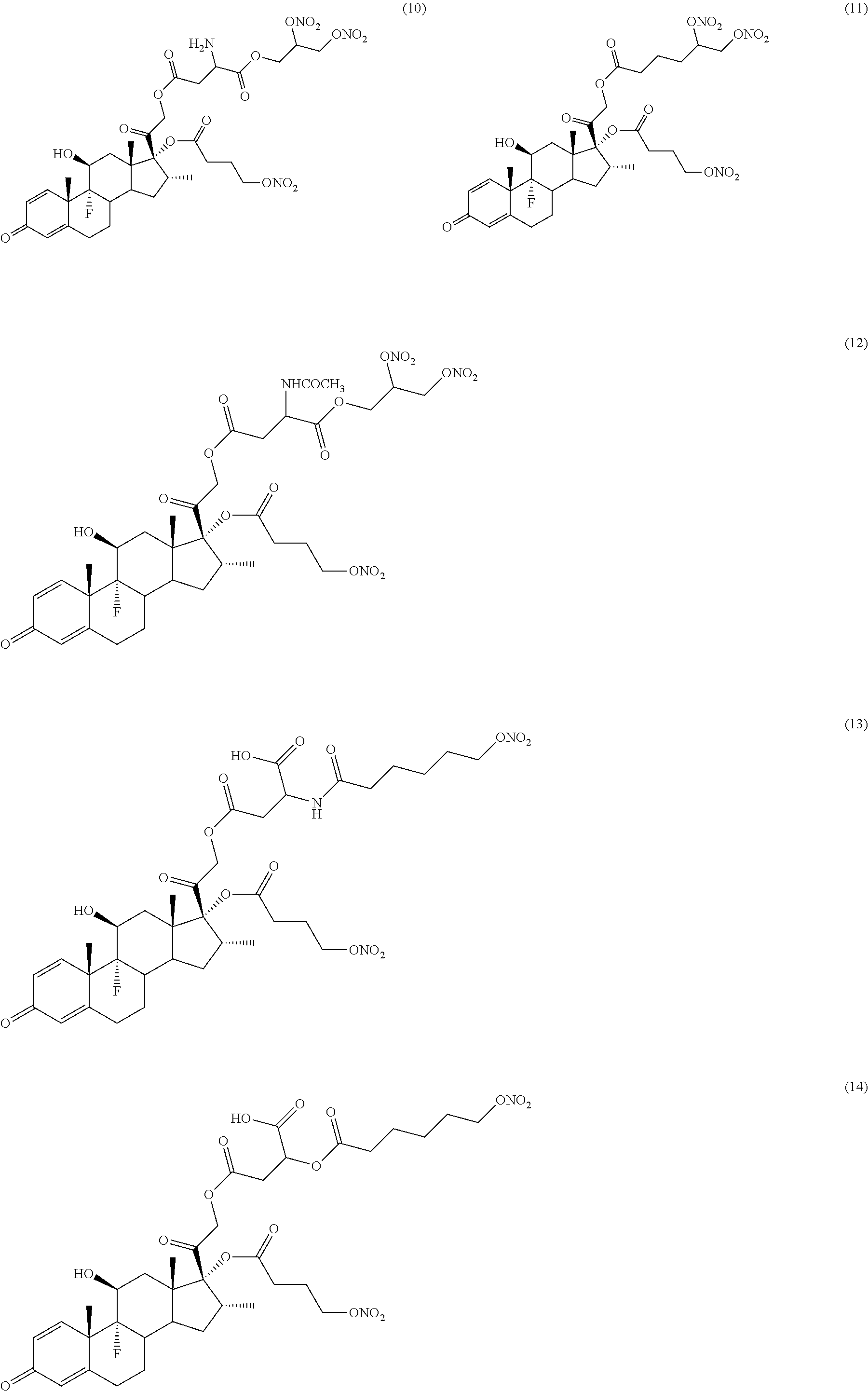

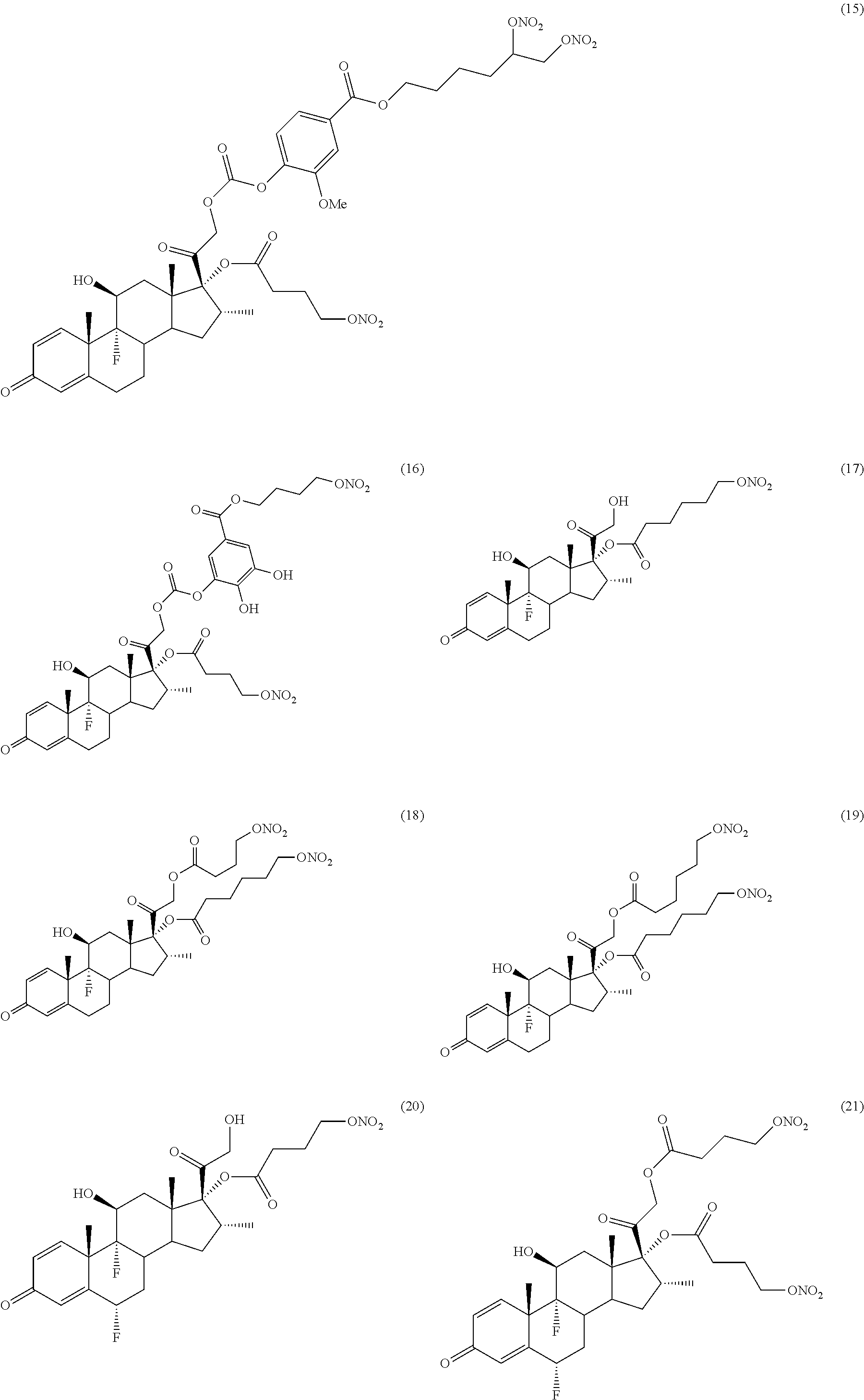

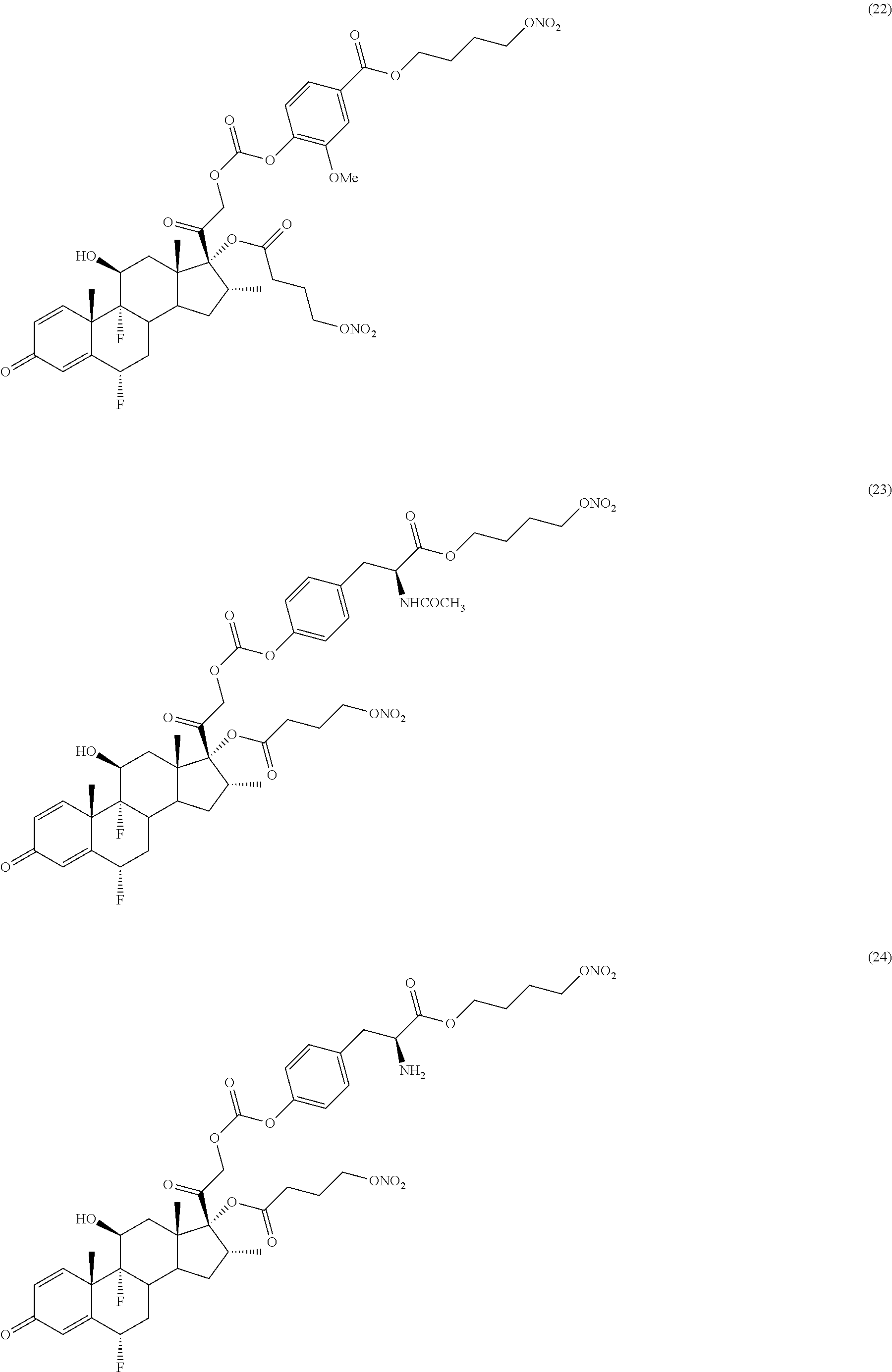

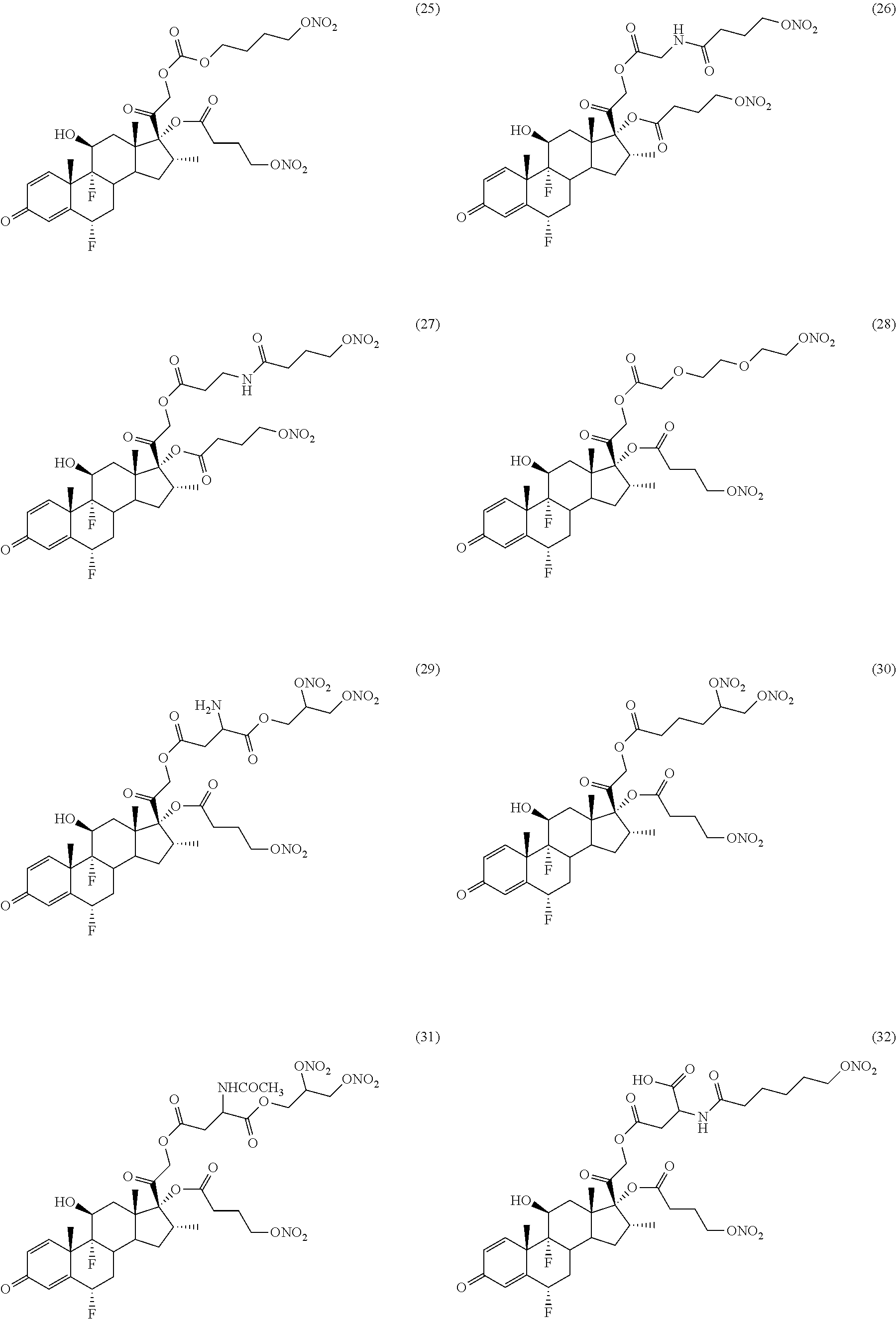

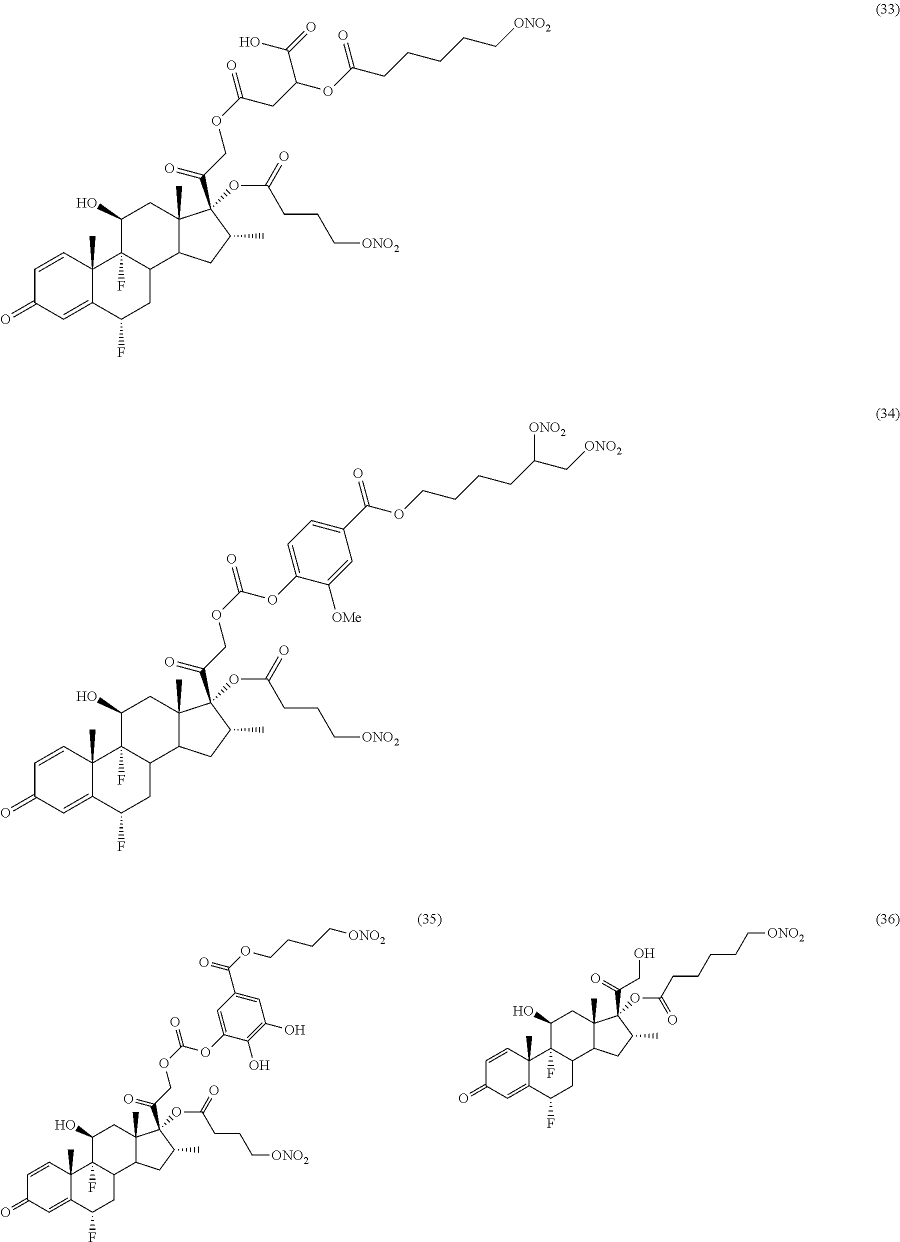

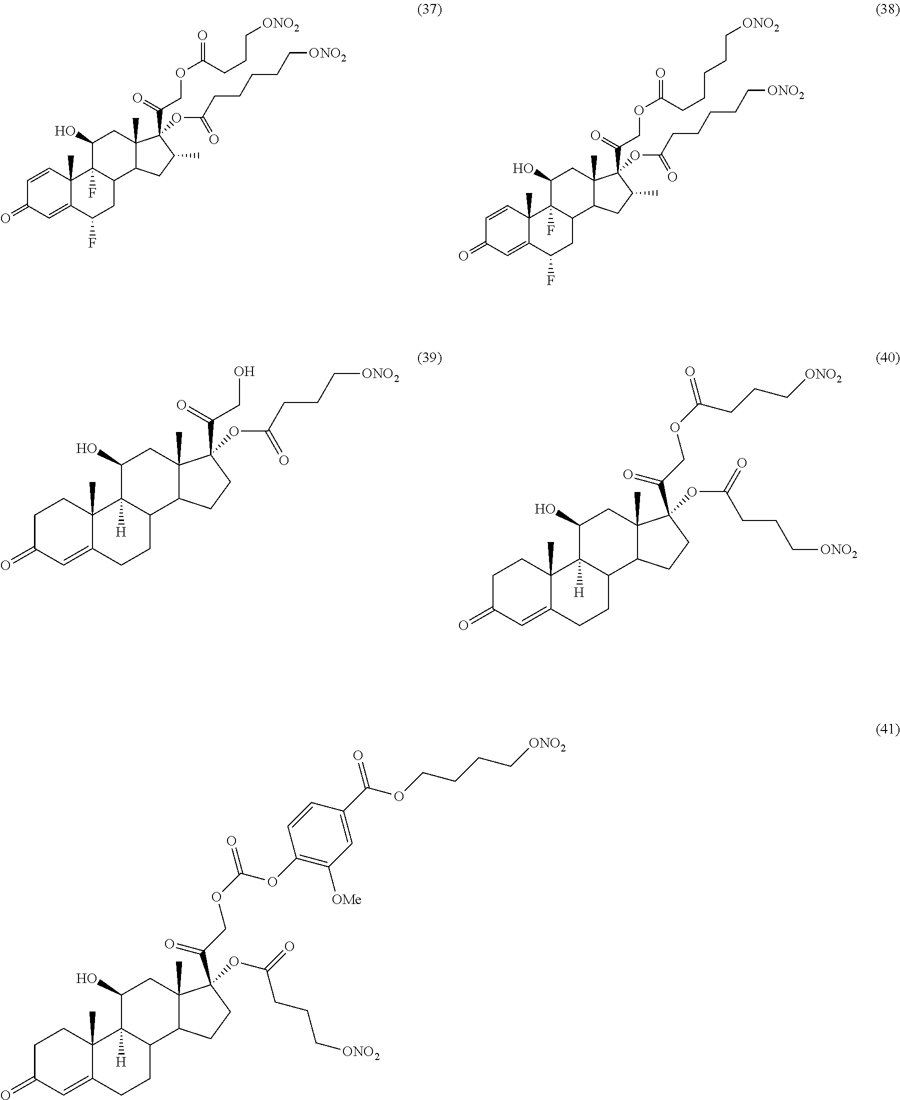

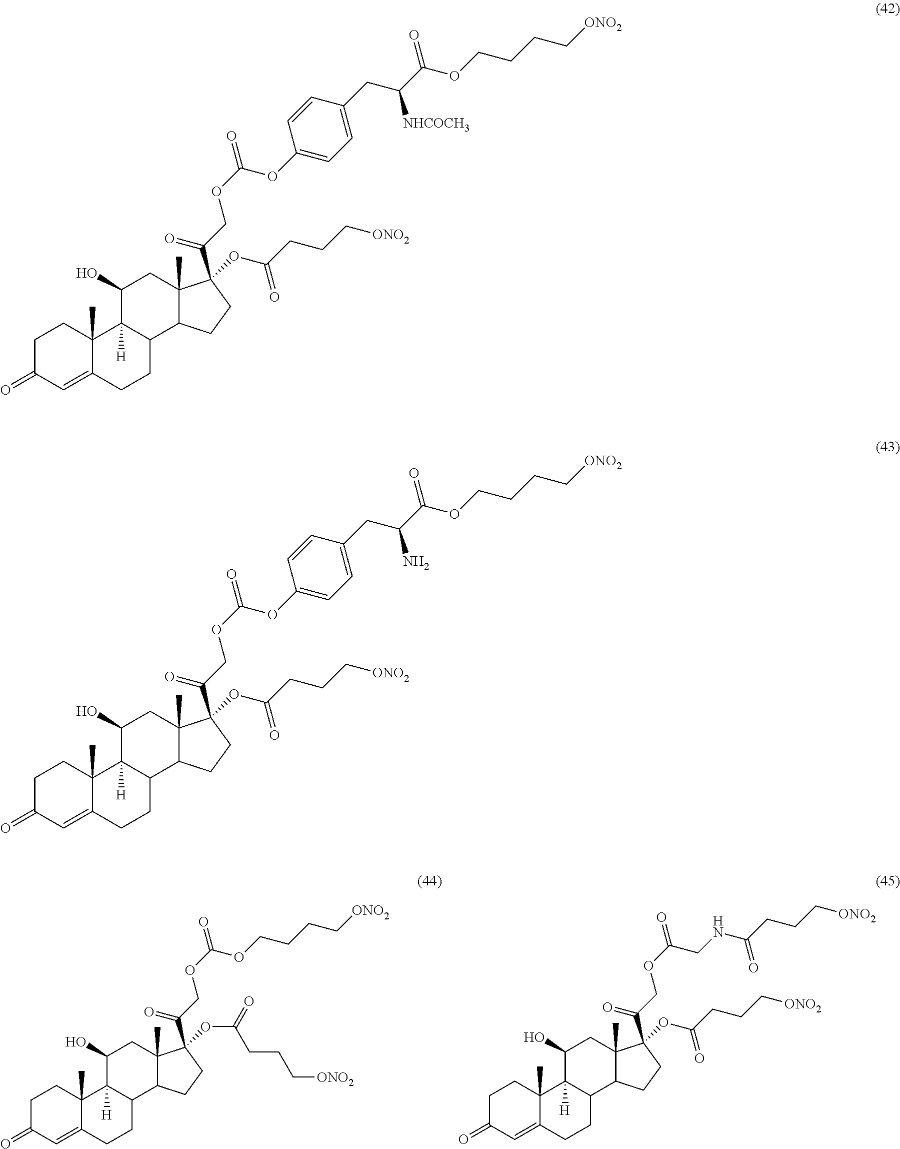

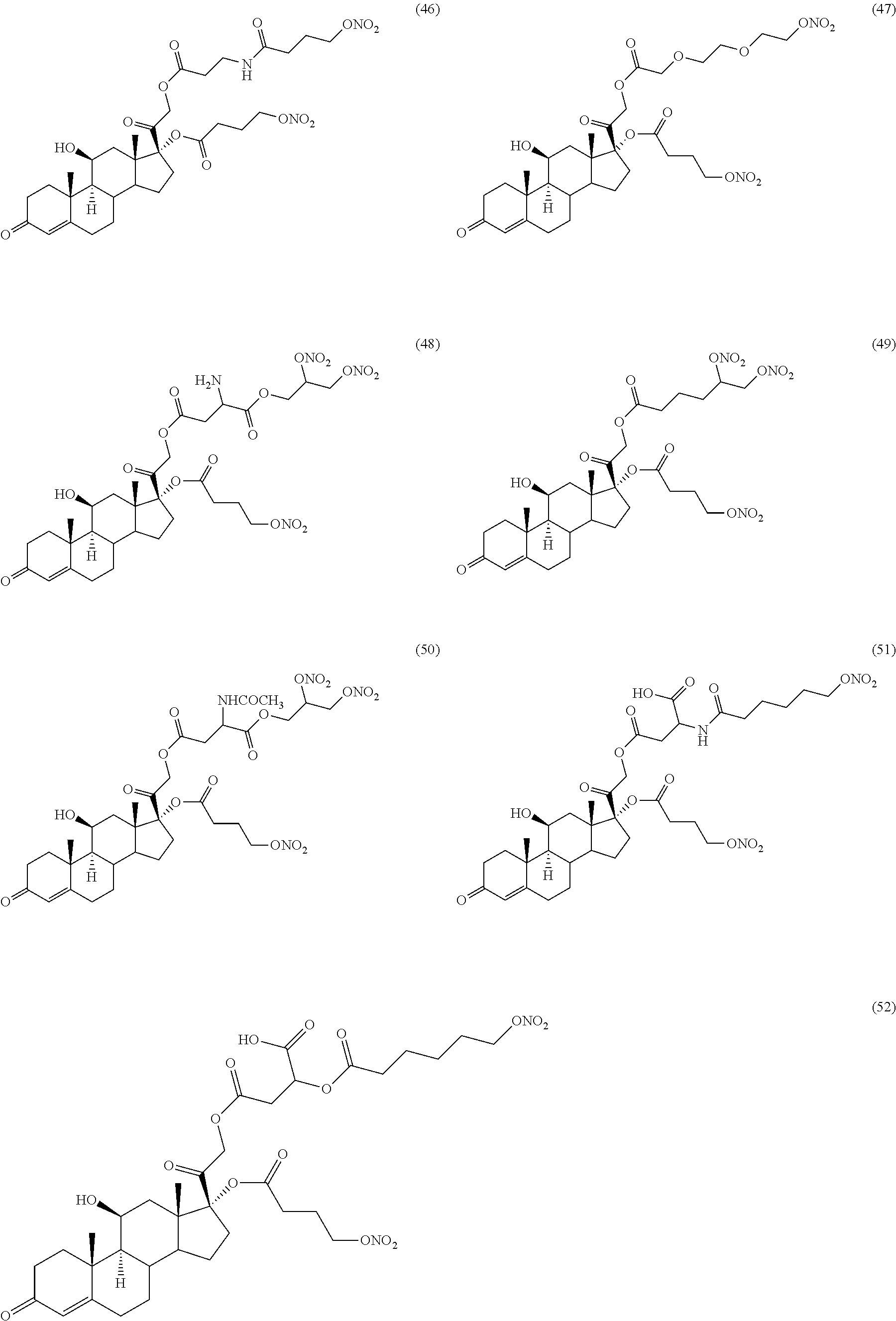

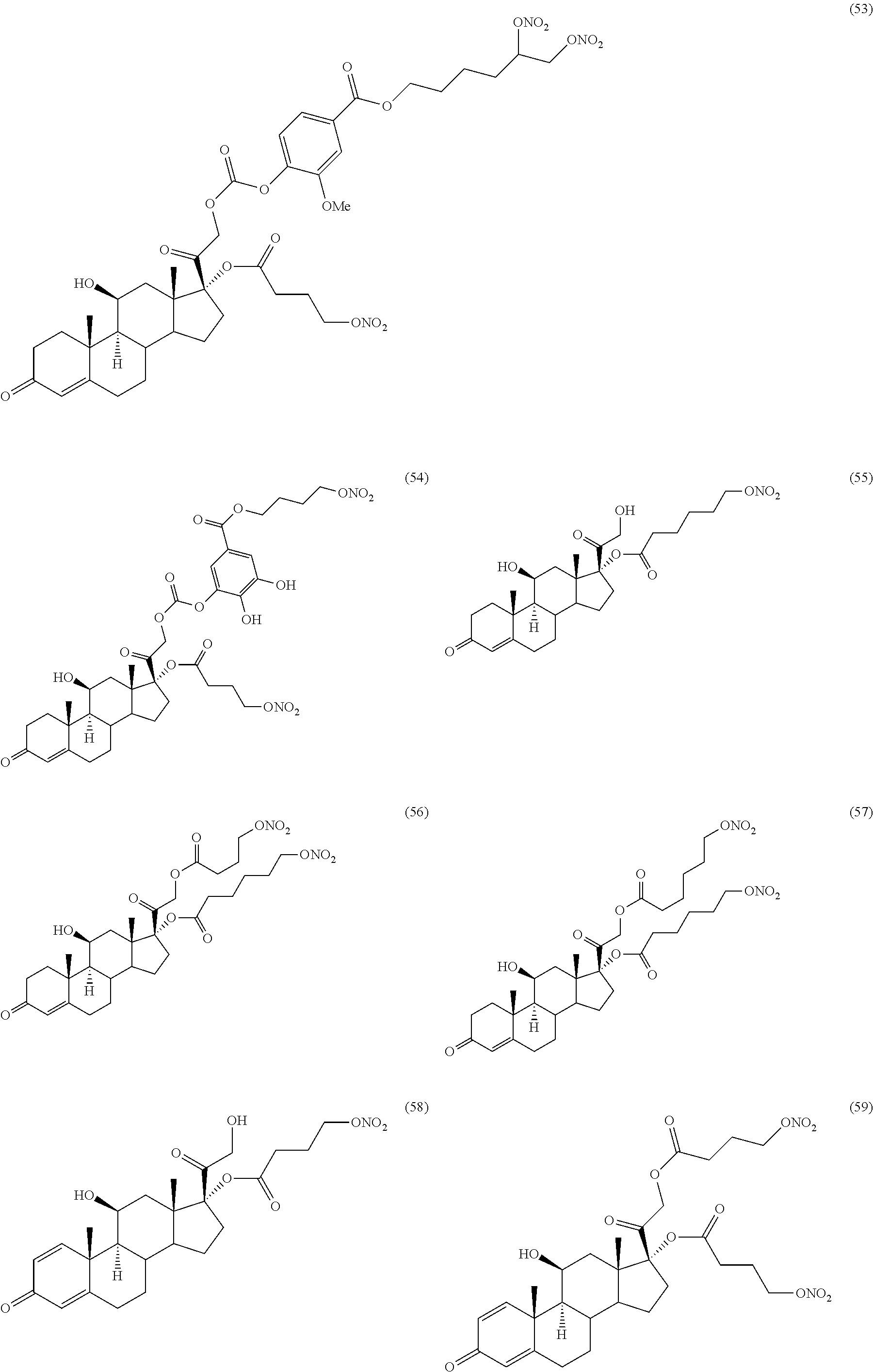

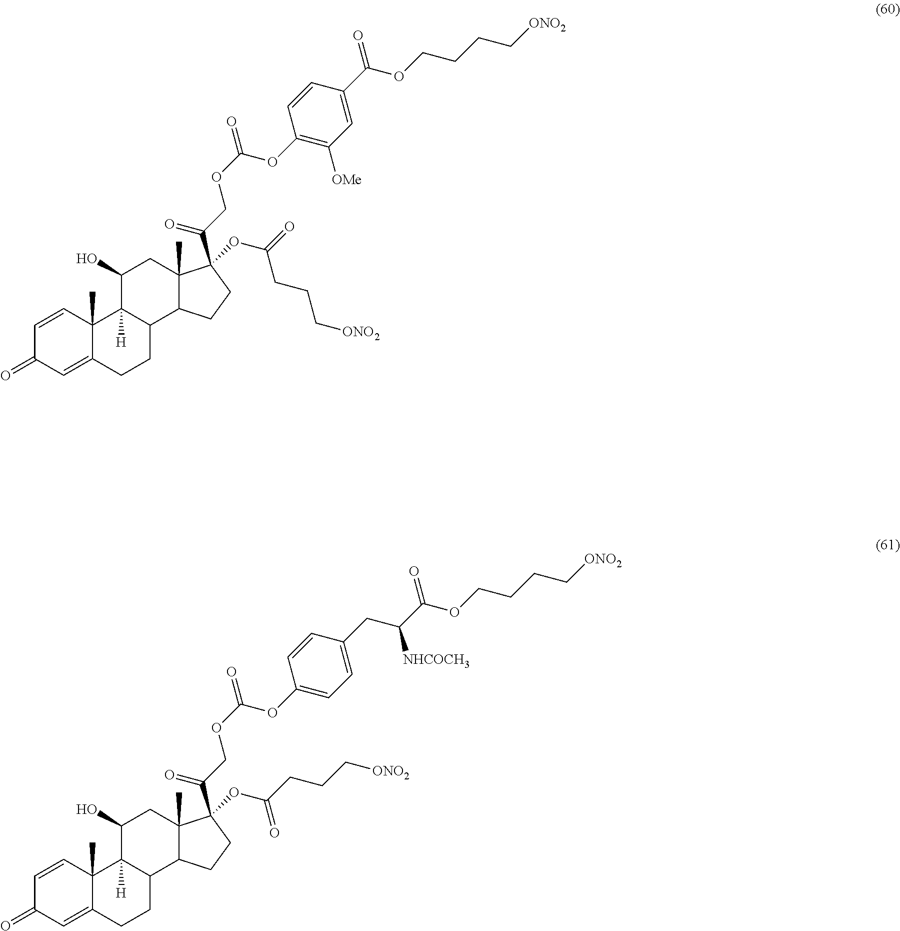

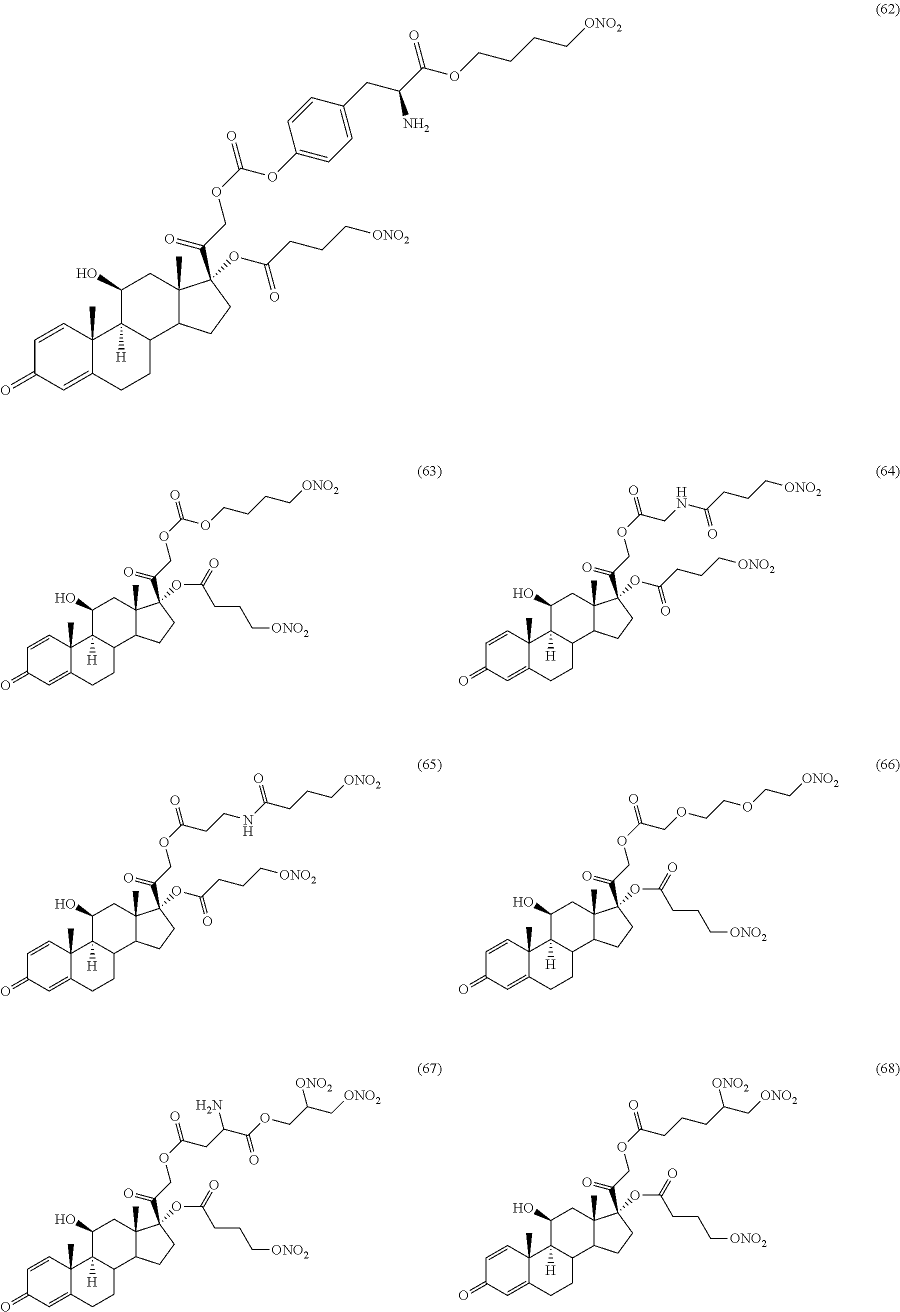

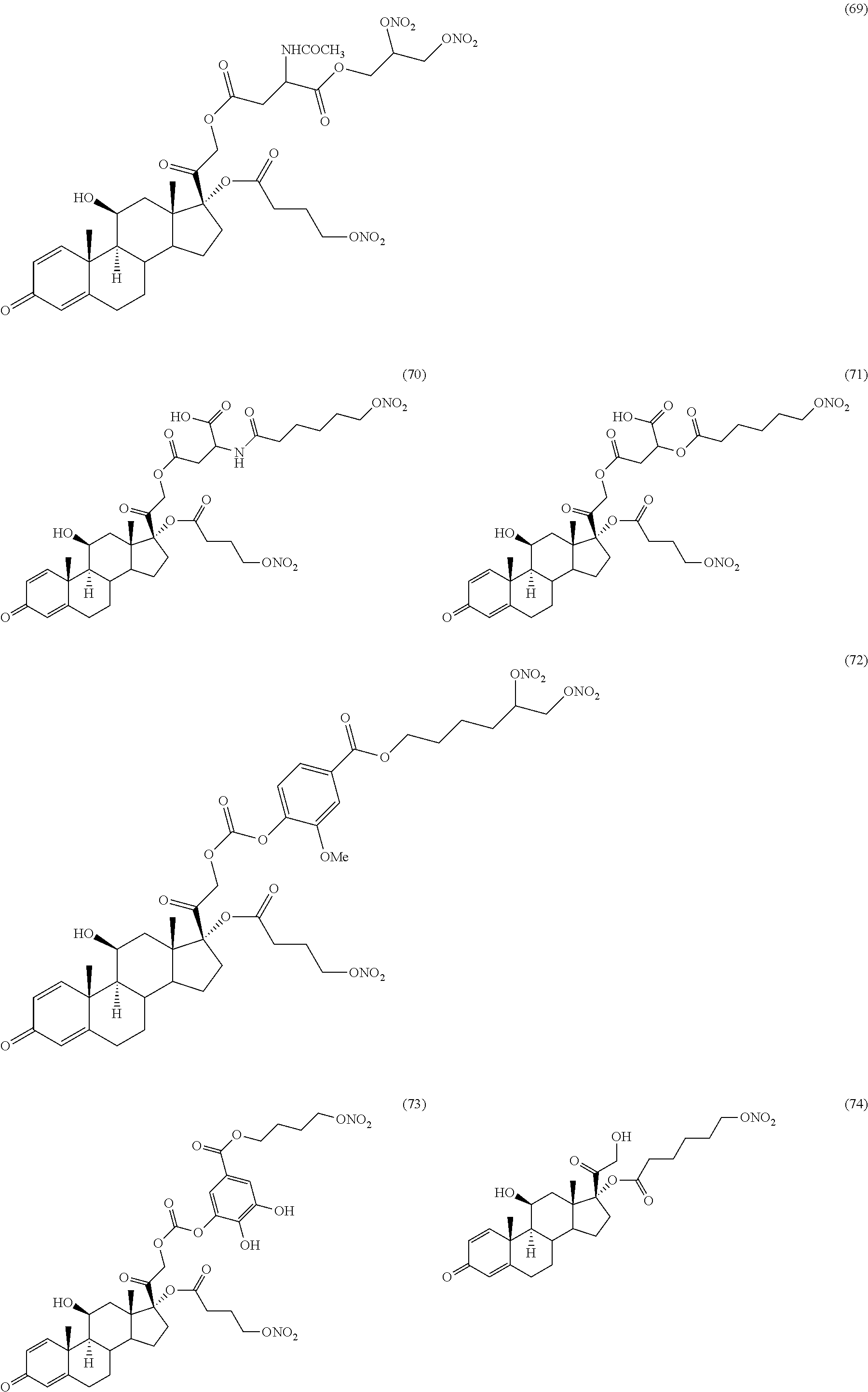

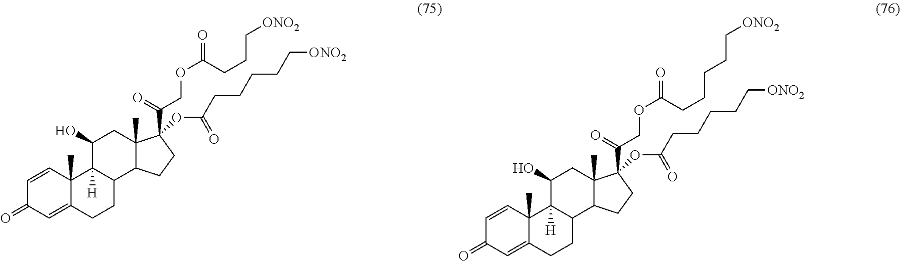

In another aspect of the invention, there is provided a compound of formula (I) for the use in the treatment of rheumatic diseases, renal and bronchial pathologies, ocular and dermatological diseases, autoimmune diseases, tumoral processes, also in combination with chemotherapeutic and/or radiotherapeutic treatments, in neurodegenerative diseases, for example in spinal lesions from trauma and in the post-transplant therapy. Furthermore inflammatory pathologies affecting the gatrointestinal system (Crohn disease, ulcerous colitis and IBD (inflammatory bowel diseases) can be mentioned.

In yet another aspect of the invention, there is provided a pharmaceutical composition comprising an acceptable carrier and a pharmaceutically effective amount of a compound of formula (I) and/or a salt or stereoisomer thereof, or such a pharmaceutical composition in a suitable form for parenteral, oral and topic use, such as for example sublingual, inhalatory, suppository, transdermal, enema, according to the well known techniques in the art, together with the usual excipients; see for example the publication “Remington's Pharmaceutical Sciences” 15th Ed.

The amount on a molar basis of the active principle in said compositions is generally the same or lower than that of the corresponding precursor drug.

The daily administrable doses are those of the precursor drugs, or optionally lower. The precursor daily doses can be found in the publications of the field, such for example in the “Physician's Desk reference”.

As used herein, the terms “treat,” “treating” or “treatment” includes preventative (e.g., prophylactic) and palliative treatment.

As used herein, the term “pharmaceutically acceptable” means the carrier, diluent, excipients and/or salt must be compatible with the other ingredients of the formulation and not deleterious to the recipient thereof.

As used herein, the term “alkyl” means a straight or branched chain saturated hydrocarbon. Exemplary alkyl groups include but are not limited to methyl, ethyl, propyl, isopropyl, n-butyl, sec-butyl, tert-butyl, pentyl, isopentyl, neopentyl, tert-pentyl, 1-methylbutyl, methylbutyl, 3-methylbutyl, hexyl, isohexyl, heptyl, octyl and the like.

The term “C₁-C₁₀ alkylene” as used herein refers to branched or straight alkylene groups including methylene, ethylene, n-propylene, isopropylene, n-butylene, isobutylene, t-butylene, pentylene, hexylene, octylene and the like.

### Synthesis Procedure

In general the term “amino protecting group” as used herein refers to Boc, Fmoc or those described in T. W. Greene “Protective groups in organic synthesis”, Wiley-Interscience, 2007, 4th edition.

The term “carboxylic protecting group” as used herein refers to tert-butyl ester and those described in T. W. Greene “Protective groups in organic synthesis”, Wiley-Interscience, 2007, 4th edition.

The term “diol protecting group” as used herein refers to acetal, such as p-methoxybenzylidene, butylidene, and those described in T. W. Greene “Protective groups in organic synthesis”, Wiley-Interscience, 2007, 4nd edition;

The term “hydroxyl protecting group” as used herein refers to silyl ethers, such as trimethylsilyl, tert-butyl-dimethylsilyl or trityl and those described in T. W. Greene “Protective groups in organic synthesis”, Wiley-Interscience, 2007, 4th edition.

1) The compound of general formula (I) as above defined wherein R₁ is H, R₂, R₃, R₄, R₄ₐ, R₅, R₆, R₆ₐ, R₇, R₇ₐ, R₈, R₈ₐ, R₉, R₁₀, R₁₀ₐ are as above defined, can be obtained:
1.1) by reacting a compound of formula (IIa), i.e. the precursor corticosteroid,

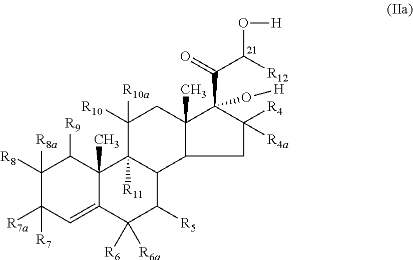

wherein R₄, R₄ₐ, R₅, R₆, R₆ₐ, R₇, R₇ₐ, R₈, R₈ₐ, R₉, R₁₀, R₁₀ₐ are as above defined
with a compound of formula (IIIa)

(R_{A}O)₃C—R₂—CH(Q)R₃ (IIIa)

wherein:
R_{A} is straight alkyl C₁-C₁₀, R₂ and R₃ are as above defined and Q is ONO₂ or Q₁, wherein Q₁ is selected from the group consisting of: a chlorine atom, a bromine atom, a iodine atom, a mesyl group or a tosyl group; the reaction is carried out in presence of an organic acid such as p-toluensulfonic acid. The reaction is carried out in an inert organic solvent such as tetrahydrofuran, dioxane, at a temperature from −20° C. and 40° C. The reaction is completed within a time range from 30 minutes to 36 hours and
   1.2) hydrolyze the ortho ester of formula (IIb) obtained in 1.1)

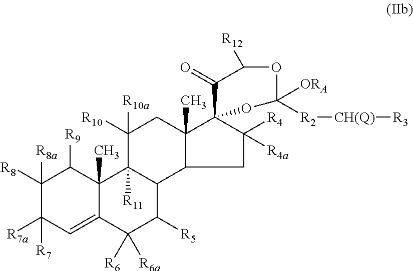

wherein R₄, R₄ₐ, R₅, R₆, R₆ₐ, R₇, R₇ₐ, R₈, R₈ₐ, R₉, R₁₀, R₁₀ₐ, R_{A} and Q are as above defined, by reacting the compound (IIb) with an organic acid such as AlCl₈, acetic acid, ossalic acid in an organic aqueous solvent such as methanol, ethanol, propanol, isopropanol at a temperature from −20° C. and 40° C. The reaction is completed within a time range from 30 minutes to 36 hours and
1.3) when Q is Q₁, by reacting the compound obtained in the step 1.2) with a nitrate source such as silver nitrate, lithium nitrate, sodium nitrate, potassium nitrate, magnesium nitrate, calcium nitrate, iron nitrate, zinc nitrate or tetraalkylammonium nitrate (wherein alkyl is C₁-C₁₀ alkyl) in a suitable organic solvent such as acetonitrile, tetrahydrofurane, methyl ethyl ketone, ethyl acetate, DMF; the reaction is carried out, in the dark, at a temperature from room temperature to the boiling temperature of the solvent. Alternatively, the reaction with AgNO₃ can be performed under microwave irradiation in solvents such acetonitrile or THF at temperatures in the range between about 100-180° C. for time range about 1-60 min. Preferred nitrate source is silver nitrate.

The compounds of formula (IIa) are commercially available

2) The compound of general formula (I) as above defined wherein R₂, R₃, R₄, R₄ₐ, R₅, R₆, R₆ₐ, R₇, R₇ₐ, R₈, R₈ₐ, R₉, R₁₀, R₁₀ₐ are as above defined, and
R₁ is selected from:
   (A) —R¹—CH(NHR²)—C(O)—O—Y
   (B) —R¹—CH(COOH)NH—C(O)—Y
   (C) —R¹—CH(COOH)—O—C(O)—Y
   (F) —(Z)—Y
   (G)

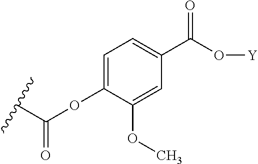

(H)

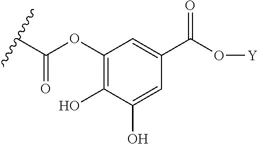

(I)

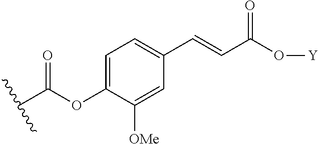

wherein
R¹ is selected from the group R^{1a}) as above defined,
R² is as above defined,
Z is —C(O)O— and
Y is as above defined,
can be synthesized:
   2.1) by reacting a compound of formula (IIc)

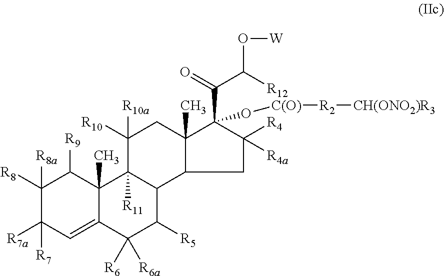

wherein R₂, R₃, R₄, R₄ₐ, R₅, R₆, R₆ₐ, R₇, R₇ₐ, R₈, R₈ₐ, R₉, R₁₀, R₁₀ₐ are as above defined and W is —H or —COCl
with a compound of the following formulae
   (A₁) W₁—R^{1a′}—CH(NHR^{2a})—C(O)—O—Y′
   (B₁) W₁—R^{1a′}—CH(COOP)NH—C(O)—Y′
   (C₁) W₁—R^{1a′}—CH(COOP)—O—C(O)—Y′
   (F₁) W₁—O—Y′
   (G₁)

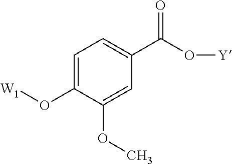

(H₁)

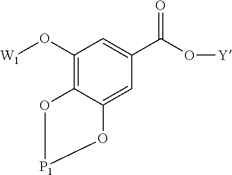

(I₁)

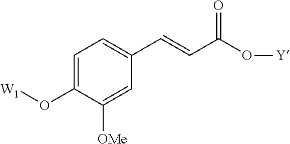

wherein
W₁ is —H or R_{B}OC(O)— wherein R_{B} is pentafluorophenyl, 4-nitrophenyl
   R^{1a′}) is selected from

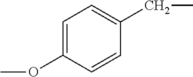

—S—CH₂—, —O—CH(CH₃)—, —O—CH₂—;
R^{2a} is —H or —C(O)CH₃ or P₂ wherein P₂ is a amino protecting group,
P is a carboxylic protecting group, P₁ is a diol protective group,
Y′ is
—R₁₃—CH(Q)R₁₄
—R₁₃—CH(Q)-(CR₁₅R₁₆)ₜ—CH(Q)R₁₄
—[(CH₂)ₒ—X]ₚ—[(CH₂)ᵣ—X]ₛ—(CH₂)_{q}—(CR₁₅R₁₆)ₜ—CH(Q)R₁₄
—[(CH₂)ₒ—X]ₚ—[(CH₂)ᵣ—X]ₛ—(CH₂)_{q}—CH(ONO₂)—(CR₁₅R₁₆)ₜ—CH(Q)R₁₄
   wherein
X, R₁₃, R₁₄, R₁₅, R₁₆, o, p, q, r, s and t are as above defined, Q is ONO₂ or Q₁, wherein Q₁ is selected from Cl, Br, I, a mesyl group or a tosyl group;
   2.1.a) The reaction of a compound of formula (IIc) wherein W is H with a compound of formula (A₁), (B₁), (C₁), (F₁), (G₁), (H₁) or (I₁) wherein W₁ is R_{B}OC(O)— is carried out in presence of a catalyst, such as DMAP or in the presence of DMAP and a Lewis acid such as Sc(OTf)₃ or Bi(OTf)₃ in an inert organic solvent such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours.
   2.1.b) The reaction of a compound of formula (IIc) wherein W is COCl with a compound of formula with a compound of formula (A₁), (B₁), (C₁), (F₁), (G₁), (H₁) or (I₁) wherein W₁ is H may be carried out in presence of an organic base such as N,N-dimethylamino pyridine (DMAP), triethylamine, pyridine. The reaction is carried out in an inert organic solvent such as N,N′-dimethylformamide, tetrahydrofuran, benzene, toluene, dioxane, a polyhalogenated aliphatic hydrocarbon at a temperature from −20° C. and 40° C. The reaction is completed within a time range from 30 minutes to 36 hours.
   2.2) when Q is Q₁, by converting the compound obtained in the step 2.1) into nitro derivative by reaction with a nitrate source according to the method described in 1.3) and
   2.3) optionally deprotecting the compounds obtained in step 2.1) or 2.2) as described in T. W. Greene “Protective groups in organic synthesis”, Wiley-Interscience, 2007, 4^{nd} edition. Trifluoroacetic acid or anhydrous inorganic acid are the preferred method for removing Boc protecting group, organic base such as piperidine is the preferred method for removing Fmoc protecting group. Aqueous or anhydrous organic or inorganic acid is the preferred method for removing t-butyl ester protecting group. Hydrochloric acid in tetrahydrofurane is the preferred method for removing acetal protecting group.

Alternatively the compound of general formula (I) as defined in 2) wherein R₁ is selected from (A), (B), (C), (F), (G), (H), (I), can be synthesized

3.1) by reacting a compound of formula (IIc) wherein R₂, R₃, R₄, R₄ₐ, R₅, R₆, R₆ₐ, R₇, R₇ₐ, R₈, R₈ₐ, R₉, R₁₀, R₁₀ₐ and W are as above defined with a compound of formula
(A₂) W₁—R^{1a′}—CH(NHR^{2a})—C(O)—O—P
(B₂) W₁—R^{1a′}—CH(COOP)—NH—R^{2a}
(C₂)

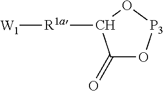

(G₂)

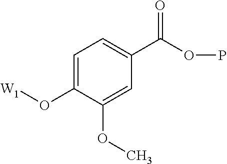

(H₂)

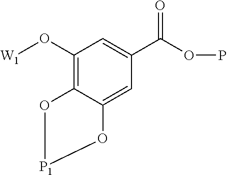

(I₂)

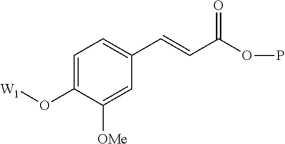

wherein
W₁ is —H or R_{B}OC(O)— wherein R_{B} is pentafluorophenyl, 4-nitrophenyl,
R^{1a′}, R^{2a}, R³, R⁴, P, P₁ are as above defined and P₃ is a alpha hydroxyl acid protecting group such as 4-oxo-1,3-dioxolane;
   3.1.a) The reaction of a compound of formula (IIc) wherein

W is H with a compound of formula (A₂), (B₂), (C₂), (G₂), (H₂), (I₂) wherein W₁ is R_{B}OC(O)— is carried out according to the method described in 2.1.a).

3.1.b) The reaction of a compound of formula (IIc) wherein The reaction of a compound of formula (IIc) wherein W is COCl with a compound of formula (A₂), (B₂), (C₂), (G₂), (H₂), (I₂) wherein W₁ is H is carried out according to the method described in 2.1.b),
and
3.2) deprotecting the compounds obtained in step 3.1) as described in T. W. Greene “Protective groups in organic synthesis”, Wiley-Interscience, 2007, 4^{nd} edition, hydrochloric acid or anhydrous inorganic acid are the preferred method for removing alpha hydroxy acid protecting group,
and
3.3) by reacting a compound of formula (IId) obtained in the step 3.2)

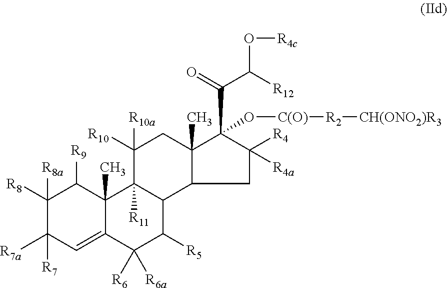

wherein R₂, R₃, R₄, R₄ₐ. R₅, R₆, R₆ₐ, R₇, R₇ₐ, R₈, R₈ₐ, R₉, R₁₀ and R₁₀ₐ are as above defined and R_{4c} is a radical selected from the following meaning
(A₃) —R^{1a}—CH(NHR^{2a})—C(O)—OH
(B₃) —R^{1a}—CH(COOP)—NH₂
(C₃) —R^{1a}—CH(COOH)—OH
(G₃)

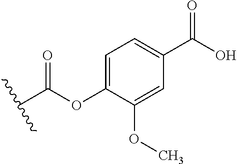

(H₃)

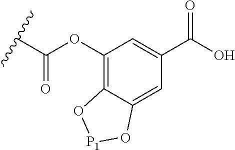

(I₃)

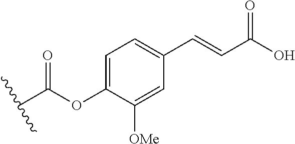

wherein
R¹ is selected from the group R^{1a}) as above defined, R^{2a} is as above defined,
with a compound of formula

W₂—R₁₃—CH(Q)R₁₄ (VIa)

W₂—R₁₃—CH(Q)-(CR₁₅R₁₆)ₜ—CH(Q)R₁₄ (VIb)

W₂—[(CH₂)ₒ—X]ₚ—[(CH₂)ᵣ—X]ₛ—(CH₂)_{q}—(CR₁₅R₁₆)ₜ—CH(Q)R₁₄ (VIc)

W₂—[(CH₂)ₒ—X]ₚ—[(CH₂)ᵣ—X]ₛ—(CH₂)_{q}—CH(Q)-(CR₁₅R₁₆)ₜ—CH(Q)R₁₄ (VId)

wherein W₂ is selected from HO—, Cl, Br, I, —COOH, —COCl, —C(O)OR_{B} wherein R_{B} is as above defined;
W₂ is —OH, Cl, Br, I when R_{4c} is selected from (A₃), (G₃), (H₃), (I₃) or W₂ is —COOH, —C(O)OR_{B}, —CO—Cl when R_{4c} is selected from (B₃), (C₃);
   3.3. a) the reaction of the compound of formula (IId) wherein R_{4c} is selected from (A₃), (G₃), (H₃), (I₃), with a compound of formula (VIa), (VIb), (VIc) or (VId) wherein W₂ is Cl, Br, I is carried out in the presence of a organic base such as 1,8-diazabiciclo[5.4.0]undec-7-ene (DBU), N,N-diisopropylethyl amine, diisopropylamine or an inorganic base such as alkaline-earth metal carbonate or hydroxide, potassium carbonate, cesium carbonate, in an inert organic solvent such as N,N′-dimethylformamide, tetrahydrofuran, acetone, methyl ethyl ketone, acetonitrile, a polyhalogenated aliphatic hydrocarbon at a temperature from −20° C. and 40° C., preferably from 5° C. to 25° C. The reaction is completed within a time range from 1 to 8 hours. When W₃ is chosen among chlorine or bromine the reaction is carried out in presence iodine salts such as KI.
   3.3.b) the reaction of a compound of formula (IId) wherein R_{4c} is a radical selected (A₃), (G₃), (H₃), (I₃), with a compound of formula (VIa), (VIb), (VIc) or (VId) wherein W₂ is —OH is carried out in the presence of a condensing agent such as dicyclohexylcarbodiimide (DCC), N′-(3-dimethyl aminopropyl)-N-ethylcarbodiimide hydrochloride (EDAC), N,N′-carbonyldiimidazole (CDI), optionally in the presence of a base, for example DMAP, in an inert organic solvent dry such as N,N′-dimethylformamide, tetrahydrofuran, benzene, toluene, dioxane, a polyhalogenated aliphatic hydrocarbon at a temperature from −20° C. and 50° C. The reaction is completed within a time range from 30 minutes to 36 hours;
   3.3.c) the reaction of a compound of formula (IId) wherein R_{4c} is (B₃) or (C₃) with a compound of formula (VIa), (VIb), (VIc) or (VId) wherein W₂ is —COOH is carried out according to the method described in 3.3.b) or in presence of other condensing reagents such as O-(7-azabenzotriazol-1-yl)-N,N,N,N′-tetramethyluronium hexafluorophosphate (HATU);
   3.3.d) The reaction of a compound of formula (IId) wherein R_{4c} is (B₃) or (C₃) with a compound of formula (VIa), (VIb), (VIc) or (VId) wherein W₂ is —COCl may be carried out according to the method described in 2.1.b);
   3.3.e) the reaction of a compound of formula (IId) wherein R_{4c} is (B₃) or (C₃) with a compound of formula (VIa), (VIb), (VIc) or (VId) wherein W₂ is R_{B}OC(O)— is carried out according to the method described in 2.1.a), and
   3.4) when Q is Q₁, by converting the compound obtained in the step 3.3) into nitro derivative according to the method described in 1.3)
   and
   3.5) deprotecting the compounds obtained in step 3.3) or 3.4) as described in T. W. Greene “Protective groups in organic synthesis”, Wiley-Interscience, 2007, 4^{nd} edition.
   4) The compound of general formula (I) as above defined wherein R₂, R₃, R₄, R₄ₐ, R₅, R₆, R₆ₐ, R₇, R₇ₐ, R₈, R₈ₐ, R₉, R₁₀, R₁₀ₐ are as above defined and r1 is selected from:
(A) —R¹—CH(NHR²)—C(O)—O—Y
(B) —R¹—CH(COOH)NH—C(O)—Y
(C) —R¹—CH(COOH)—O—C(O)—Y
(D) —C(O)CH(R³)—NH—C(O)—Y
(E) —C(O)CH₂—CH(R⁴)—NH—C(O)—Y
(F) —(Z)—Y
   wherein
R¹ is selected from the group R^{1b}) as above defined,
R², R³, R⁴ and Y are as above defined,
Z is —C(O)—,
can be synthesized:
   4.1) by reacting a compound of formula (IIc) as above defined wherein R₂, R₃, R₄, R₄ₐ, R₅, R₆, R₆ₐ, R₇, R₇ₐ, R₈, R₈ₐ, R₉, R₁₀, R₁₀ₐ are as above defined and W is H, with a compound of formula:
(A₄) W₃—R¹—CH(NHR^{2a})—C(O)—O—Y′
(B₄) W₃—R¹—CH(COOP)NH—C(O)—Y′
(C₄) W₃—R¹—CH(COOP)—O—C(O)—Y′
(D₁) W₃—C(O)CH(R³)—NH—C(O)—Y′
(E₁) W₃—C(O)CH₂—CH(R⁴)—NH—C(O)—Y′
(F₂) W₃—(Z)—Y′
   wherein W₃ is HO— or R_{B}O— wherein R_{B} is as above defined,
R₁, R₂ₐ, R₃, R₄, P and Y′ are as above defined;
   4.1.a) The reaction of a compound of formula (IIc) wherein W is H with a compound of formula (A₄), (B₄), (C₄), (D₁), (E₁) or (F₂) wherein W₃ is R_{B}O— is carried out as reported in 2.1.a);
   4.1.b) the reaction of a compound of formula (IIc) wherein W is H with a compound of formula (A₄), (B₄), (C₄), (D₁), (E₁) or (F₂) wherein W₃ is HO—, is carried out as reported in 3.3.b);
   and
   4.2) when Q is Q₁, by reacting the compound obtained in the step 4.1) with a nitrate source according to the method described in 1.3)
   and
   4.3) optionally deprotecting the compounds obtained in step 4.1) or 4.2) as described in T. W. Greene “Protective groups in organic synthesis”, Wiley-Interscience, 2007, 4^{nd} edition.

Alternatively the compound of general formula (I) as defined in 4), wherein R₁ is selected from (A), (B), (C), (F), (G), (H), (I), can be synthesized

4.4) by reacting a compound of formula (IIc) as above defined with a compound of formula
(A₅) W₃—R¹—CH(NHR^{2a})—C(O)—O—P
(B₅) W₃—R¹—CH(COOH)—NH—R^{2a}
(C₅)

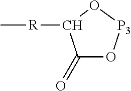

(D₂) W₃—C(O)—CH(R³)—NH—R^{2a}
(E₂) W₃—C(O)—CH₂—CH(R⁴)—NH—R^{2a}
   wherein:
W₃, R¹, R^{2a}, R³, R⁴, P and P₃ are as above defined;
   4.4.a) the reaction of a compound of formula (IIc) with a compound of formula (A₅), (B₅), (C₅), (D₂) or (E₂) wherein W₃ is HO—, is carried out according to the method described in 4.1.b),
   4.4.b) the reaction of a compound of formula (IIc) wherein W is H with a compound of formula (A₅), (B₅), (C₅), (D₂) or (E₂) wherein W₃ is R_{B}O— is carried out according to the method described in 4.1.a),
   and
   4.5) deprotecting the compounds obtained in step 4.4.a) or 4.4.b) as described in T. W. Greene “Protective groups in organic synthesis”, Wiley-Interscience, 2007, 4^{nd} edition, and
   4.6) by reacting a compound of formula (IIe) obtained in the step 4.5)

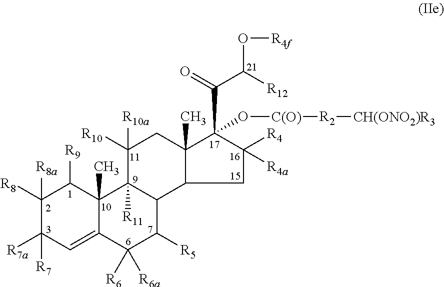

wherein R₂, R₃, R₄, R₄ₐ, R₅, R₆, R₆ₐ, R₇, R₇ₐ, R₈, R₈ₐ, R₉, R₁₀, R₁₀ₐ are as above defined and R_{4f} is a radical selected from:
(A₆) —R¹—CH(NHR^{2a})—C(O)OH
(B₆) —R¹—CH(COOP)—NH₂
(C₆) —R¹—CH(COOH)—OH
(D₃) —C(O)—CH(R³)—NH₂
(E₃) —C(O)—CH₂—CH(R⁴)—NH₂
   wherein R¹ is selected from the group R^{1b}) as above defined,
R^{2a}, R³, R⁴ and P are as above defined,
with a compound of formula

W₂—R₁₃—CH(Q)R₁₄ (VIa)

W₂—R₁₃—CH(Q)-(CR₁₅R₁₆)ₜ—CH(Q)R₁₄ (VIb)

W₂—[(CH₂)ₒ—X]ₚ—[(CH₂)ᵣ—X]ₛ—(CH₂)_{q}—(CR₁₅R₁₆)ₜ—CH(Q)R₁₄ (VIc)

W₂—[(CH₂)ₒ—X]ₚ—[(CH₂)ᵣ—X]ₛ—(CH₂)_{q}—CH(Q)-(CR₁₅R₁₆)ₜ—CH(Q)R₁₄ (VId)

wherein
W₂ is HO—, Cl, Br, I when R_{4f} is (A₆) or W₂ is —COOH, —C(O)OR_{B} or —COCl when R_{4f} is (B₆), (C₆), (D₃) or (E₃);
   4.6.a) the reaction of the compound of formula (IIe) wherein R_{4f} is (A₆), with a compound of formula (VIa), (VIb), (VIc), (VId) wherein W₂ is Cl, Br, I, is carried out according to the method described in 3.3. a);
   4.6.b) the reaction of the compound of formula (IIe) wherein R_{4f} is (B₆), (C₆), (D₃) or (E₃) with a compound of formula (VIa), (VIb), (VIc), (VId) wherein W₂ is OH, is carried out according to the method described in 2.1.c).
   4.6.c) the reaction of the compound of formula (IIe) wherein R_{4f} is (B₆), (C₆), (D₃) or (E₃) with a compound of formula (VIa), (VIb), (VIc), (VId) wherein W₂ is COOH is carried out according to the method described in 3.3.c);
   4.6.d) The reaction of the compound of formula (IIe) wherein R_{4f} is (B₆), (C₆), (D₃) or (E₃) with a compound of formula (VIa), (VIb), (VIc), (VId) wherein W₂ is COCl may be carried out according to the method described in 2.1.b);
   4.6.e) the reaction of the compound of formula (IIe) wherein R_{4f} is (B₆), (C₆), (D₃) or (E₃) with a compound of formula (VIa), (VIb), (VIc), (VId) wherein W₂ is —C(O)OR_{B} is carried out according to the method described in 2.1.a), and
   4.7) when Q is Q₁, by reacting the compound obtained in steps 4.6.a)-4.6.e) according to the method described in 1.3) and
   4.8) deprotecting the compounds obtained in step 4.6) or 4.7) as described in T. W. Greene “Protective groups in organic synthesis”, Wiley-Interscience, 2007, 4^{nd} edition.

### 5) Preparation of Compound (IIC)

The compounds of formula (IIc) wherein R₂, R₃, R₄, R₄ₐ, R₅, R₆, R₆ₐ, R₇, R₇ₐ, R₈, R₈ₐ, R₉, R₁₀, R₁₀ₐ are as above defined and W is —COCl are prepared starting from the compounds obtained in 1.3), according to methods known in the literature.

### 6) Preparation of Compound (IIIa)

The compounds of formula (IIIa) wherein R_{A}, R₂, R₃ are as above defined and Q is Qᵢ are commercially available or can be obtained according to methods known in the literature.

The compounds of formula (IIIa) wherein R_{A}, R₂, R₃ are as above defined and Q is ONO₂ can be obtained by reacting the compound (IIIa) wherein Q is Q₁ with a nitrate source as above described.

### 7) Preparation of the Following Compounds

(A₁) W₁—R^{1a′}—(NHR^{2a})—C(O)—O—Y′
(B₁) W₁—R^{1a′}—CH(COOP)NH—C(O)—Y′
(C₁) W₁—R^{1a′}—CH(COOP)—O—C(O)—Y′
(A₄) W₃—R¹—CH(NHR^{2a})—C(O)—O—Y′
(B₄) W₃—R¹—CH(COOP)NH—C(O)—Y′
(C₄) W₃—R¹—CH(COOP)—O—C(O)—Y′
(D₁) W₃—C(O)CH(R³)—NH—C(O)—Y′
(E₁) W₃—C(O)CH₂—CH(R⁴)—NH—C(O)—Y′

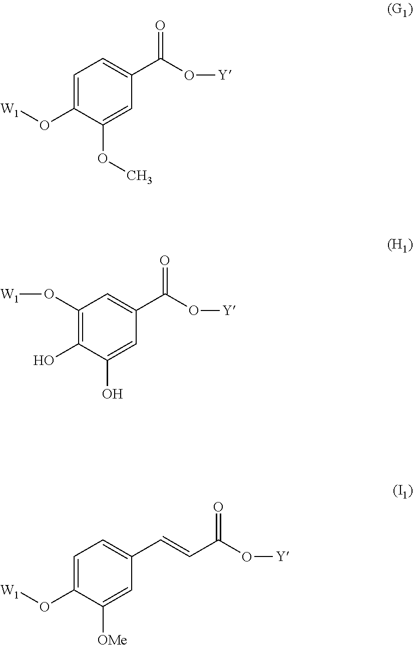

wherein
W₁ is H, W₃ is —OH,
R^{1a′}, R^{2a}, R³, R⁴, P and Y′ are as above defined and R¹ is selected from the group R^{1b}) as above defined, can be synthesized
   7.1) by reacting a compound of formula
(A₇) P₄—R^{1a′}—CH(NHR^{2a})—C(O)—OH
(A₈) PO—R¹—CH(NHR^{2a})—C(O)—OH

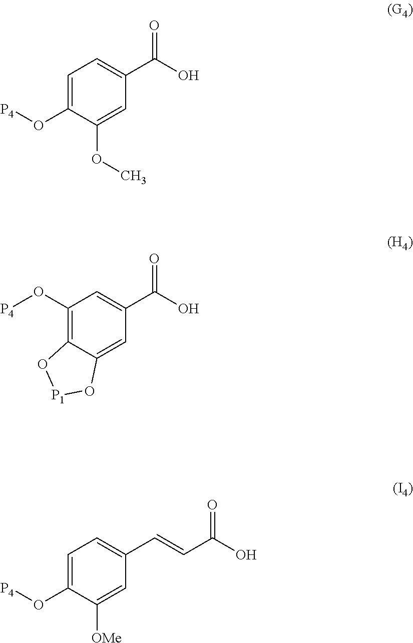

wherein
P, P₁, R^{1a′}, R^{2a} are as above defined,
R¹ is selected from the group R^{1b}) as above defined,
P₄ is a hydroxyl protecting group,
with a compound of formula

W₂—R₁₃—CH(Q)R₁₄ (VIa)

W₂—R₁₃—CH(Q)-(CR₁₅R₁₆)ₜ—CH(Q)R₁₄ (VIb)

W₂—[(CH₂)ₒ—X]ₚ—[(CH₂)ᵣ—X]ₛ—(CH₂)_{q}—(CR₁₅R₁₆)ₜ—CH(Q)R₁₄ (VIc)

W₂—[(CH₂)ₒ—X]ₚ—[(CH₂)ᵣ—X]ₛ—(CH₂)_{q}—CH(Q)CR₁₅R₁₆)ₜ—CH(Q)R₁₄ (VId)

wherein
Q, X, o, p, r, s, t, R₁₃, R₁₄, R₁₅, R₁₆ are as above defined, W₂ is HO—, Cl, Br, I,
   7.1.a) the reaction of a compound of formula (A₇), (A₈) (G₄), (H₄), (I₄ with a compound of formula (VIa) (VIb), (VIc), (VId) wherein W₂ is Cl, Br, I is carried out according to the method described in 3.3.a)
   7.1.b) The reaction of a compound of formula (A₇), (A₈) (G₄), (H₄), (I₄) with a compound of formula (VIa) (VIb), (VIc), (VId) wherein W₂ is OH is carried out according to the method described in 2.1.c).
   7.2) or by reaction a compound of formula
(B₇) P₄—R^{1a′}—CH(COOP)—NH₂
(C₇) P₄—R^{1a′}—CH(COOH)—OH
(D₄) POC(O)—CH(R³)—NH₂
(E₄) POC(O)—CH₂—CH(R⁴)—NH₂
(B₈) PO—R¹—CH(COOP)—NH₂
(C₈) PO—R¹—CH(COOH)—OH
   wherein
P, R^{1a′}, R³, R⁴ and P are as above defined and
P₄ is a hydroxyl protecting group,
R¹ is selected from the group R^{1b}) as above defined, with a compound of formula

W₂—R₁₃—CH(Q)R₁₄ (VIa)

W₂—R₁₃—CH(Q)-(CR₁₅R₁₆)ₜ—CH(Q)R₁₄ (VIb)

W₂—[(CH₂)ₒ—X]ₚ—[(CH₂)ᵣ—X]ₛ—(CH₂)_{q}—(CR₁₅R₁₆)ₜ—CH(Q)R₁₄ (VIc)

W₂—[(CH₂)ₒ—X]ₚ—[(CH₂)ᵣ—X]ₛ—(CH₂)_{q}—CH(Q)-(CR₁₅R₁₆)ₜ—CH(Q)R₁₄ (VId)

wherein
Q, X, o, p, r, s, t, R₁₃, R₁₄, R₁₅, R₁₆ are as above defined, W₂ is —COOH, —COCl or R_{B}OC(O)— wherein R_{B} is as above defined;
   7.2.a) the reaction of a compound of formula (B₇), (B₈), (C₇), (C₈), (D₄), (E₄) with a compound of formula (VIa), (VIb), (VIc), (VId) wherein W₂ is COOH is carried out according to the method described in 3.3.c),
   7.2.b) the reaction of a compound of formula B₇), (B₈), (C₇), (C₈), (D₄), (E₄) with a compound of formula (VIa), (VIb), (VIc), (VId) wherein W₂ is —COCl is carried out according to the method described in 2.1.b).
   7.2.c) the reaction of a compound of formula B₇), (B₈), (C₇), (C₈), (D₄), (E₄)) with a compound of formula (VIa), (VIb), (VIc), (VId) wherein W₂ is R_{B}OC(O)— is carried out according to the method described in 2.1.a), and
   7.3) when Q is Q₁, by reacting the compound obtained in the steps 7.1.a), 7.1.b), 7.2.a)-7.2.c) with a nitrate source according to the method described in 1.3)
   and
   7.4) deprotecting the compounds obtained in steps 6.1) and 6.2) or 6.3) as described in T. W. Greene “Protective groups in organic synthesis”, Wiley-Interscience, 2007, 4^{nd} edition. Fluoride ion is the preferred method for removing the silyl ether group.

The compounds of formula (A₇), (A₈), (B₇), (B₈), (C₇), (C₈), (D₄), (E₄), (G₄), (H₄), (I₄) are commercially available or can be obtained according to methods known in the literature

8) The compounds of formula
(A₄) W₃—R¹—CH(NHR^{2a})—C(O)—O—Y′
(B₄) W₃—R¹—CH(COOP)NH—C(O)—Y′
(C₄) W₃—R¹—CH(COOP)—O—C(O)—Y′
(D₁) W₃—C(O)CH(R³)—NH—C(O)—Y′
(E₁) W₃—C(O)CH₂—CH(R⁴)—NH—C(O)—Y′
   wherein W₃ is R_{B}O—, R¹ is selected from the group R^{1b}), R^{2a}, R³, R⁴ P and Y′ are as above defined can be synthesized according to methods known in the literature from the correspondend compounds of formula (A₄), (B₄), (C₄), (D₁), (E₁) wherein W₃ is —OH.
   9) The compounds of formula (VIa), (VIb), (VIc), (VId) are commercially available or can be obtained according to methods as known in the literature.

### EXAMPLE 1

### Synthesis of (8S,9S,10R,11S,13S,14S,17R)-11-hydroxy-17-(2-hydroxyacetyl)-10,13-dimethyl-3-oxo-6,7,8,9,10,11,12,13,14,15,16,17-dodecahydro-3H-cyclopenta[a]phenanthren-17-yl 4-nitroxybutanoate (Compound (58))

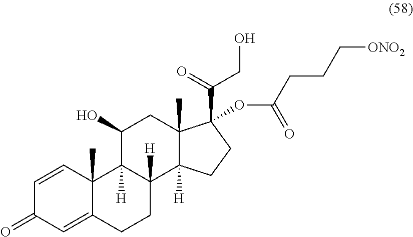

### A) (4′R,8S,9S,10R,11S,13S,14S)-2′-(3-bromopropyl)-11-hydroxy-2′-methoxy-10,13-dimethyl-7,8,9,10,11,12,13,14,15,16-decahydrospiro[cyclopenta[a]phenanthrene-17,4′-[1,3]dioxane]-3,5′(6H)-dione

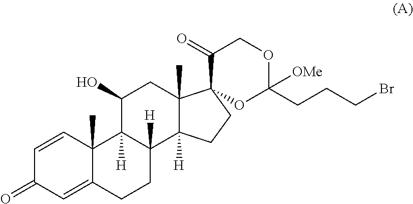

To a solution of prednisolone (1.5 g, 4.16 mmol) in toluene (28 ml) and N,N-dimethylformamide (4 ml), p-toluenesulfonic acid (cat) and trimethyl-4-bromo-orthobutyrate (1.44 ml, 8.3 mmol) were added. The reaction was stirred at room temperature for 17 hours. The mixture was poured in water (55 ml) and extracted with ethyl acetate (55×4 ml); the organic layers were dried over sodium sulfate and concentrated under reduced pressure. The residue was purified by flash chromatography, (Biotage System, SNAP Cartridge silica 100 g, eluent: gradient n-hexane/ethyl acetate 8/2 (145 ml), to n-hexane/ethyl acetate 3/7 during 1015 ml, n-hexane/ethyl acetate 3/7 (725 ml)). The product (A) (1.83 g) was obtained.

### B) (8S,9S,10R,11S,13S,14S,17R)-11-hydroxy-17-(2-hydroxy acetyl)-10,13-dimethyl-3-oxo-6,7,8,9,10,11,12,13,14,15,16, 17-dodecahydro-3H-cyclopenta[a]phenanthren-17-yl 4-bromobutanoate

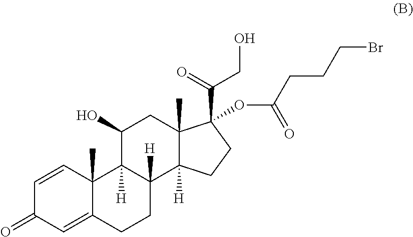

To a solution of compound (A) (1.73 g, 3.31 mmol) in methanol (59 ml), a 5% aqueous AcOH solution (11.8 ml) was added. The reaction was stirred at reflux for 5 hours. The mixture was concentrated under reduced pressure. The mixture was diluted with dichloromethane (50 ml), washed with saturated aqueous sodium carbonate (2×50 ml) and water (2×50 ml); the organic layers were dried over sodium sulfate and concentrated under reduced pressure. The residue was purified by flash chromatography, (Biotage System, SNAP Cartridge silica 100 g, eluent: gradient acetone/dichlorometane 9/1 (145 ml), to acetone/dichlorometane 25/75 during 1015 ml, acetone/dichlorometane 25/75 (435 ml)). The product (B) (1.25 g) was obtained.

### C) (8S,9S,10R,11S,13S,14S,17R)-11-hydroxy-17-(2-hydroxy acetyl)-10,13-dimethyl-3-oxo-6,7,8,9,10,11,12,13,14,15,16,17-dodecahydro-3H-cyclopenta[a]phenanthren-17-yl 4-nitroxy butanoate (Compound (58))

To a solution of compound (B) (1.24 g, 2.44 mmol) in acetonitrile (39 ml), silver nitrate (1.24 g, 7.32 mmol) was added. The reaction was heated to 130° C. for 15 minutes under microwave irradiation. The resulting mixture was cooled, filtered and the solvent was removed under reduced pressure. The residue was diluted with dichloromethane (50 ml) and washed with water (2×50 ml); the organic layer was dried over sodium sulfate and concentrated under reduced pressure. The crude was purified by flash chromatography (Biotage System, SNAP Cartridge silica 50 g, eluent: gradient acetone/dichlorometane 9/1 (75 ml), to acetone/dichlorometane 25/75 during 375 ml, acetone/dichlorometane 25/75 (375 ml)). The product (0.95 g) was obtained.

¹H-NMR: (DMSO), δ: 7.31 (1H, d, J=10.1 Hz), 6.17 (1H, d, J=10.1 Hz), 5.92 (1H, s), 5.00 (1H, t, J=6.0 Hz), 4.76 (1H, d, J=3.0 Hz), 4.50 (2H, t, J=6.3 Hz), 4.30 (1H, bs), 4.16 (1H, dd, J=18.3, 6.0 Hz), 4.05 (1H, dd, J=18.3, 6.0 Hz), 2.74 (1H, t, J=11.8 Hz), 2.56 (1H, m), 2.43 (2H, t, J=7.3 Hz), 2.30 (1H, dd, J=13.0, 2.5 Hz), 2.06 (2H, m), 1.89 (3H, m), 1.68 (3H, m), 1.54 (1H, m), 1.38 (3H, s), 1.37 (1H, m), 1.11-0.93 (2H, m), 0.84 (3H, s).

### EXAMPLE 2

### Synthesis of 4-(nitrooxy)butyl 4-((2-((8S,9S,10R,11S,13S,14S,17R)-11-hydroxy-17-(4-nitroxy butanoyloxy)-10,13-dimethyl-3-oxo-6,7,8,9,10,11,12,13,14,15,16,17-dodecahydro-3H-cyclopenta[a]phenanthren-17-yl)-2-oxoethoxy)carbonyloxy)-3-methoxybenzoate (Compound (60))

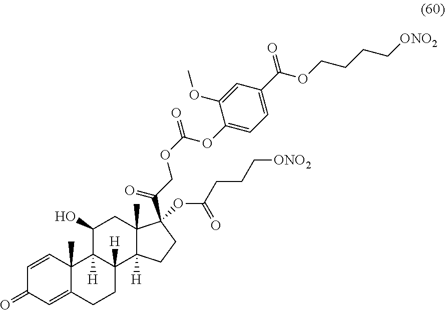

### D) (8S,9S,10R,11S,13S,14S,17R)-17-(2-(chlorocarbonyloxy)acetyl)-11-hydroxy-10,13-dimethyl-3-oxo-6,7,8,9,10,11,12,13,14,15,16,17-dodecahydro-3H-cyclopenta[a]phenanthren-17-yl 4-nitroxybutanoate

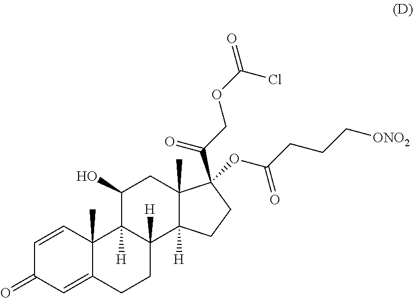

To a solution of compound (58) (0.5 g, 1.01 mmol) in tetrahydrofurane (4.8 ml), cooled at 0° C. and under N₂, a 20% toluene solution of phosgene (3.2 ml, 6.1 mmol) was added. The reaction was stirred at 0° C. for 1 hour, than at room temperature for 16 hours. The excess of phosgene was removed by heating at 40° C. for 45 minutes. The solvent was evaporated under vacuum. The crude product (D) (0.61 g) was used in the next step without any purification.

### E) 4-(nitrooxy)butyl 4-((2-((9R,10S,11S,13S,16S,17R)-9-fluoro-11-hydroxy-10,13,16-trimethyl-17-(4-(nitrooxy)butanoyloxy)-3-oxo-6,7,8,9,10,11,12,13,14,15,16,17-dodecahydro-3H-cyclopenta[a]phenanthren-17-yl)-2-oxoethoxy)carbonyloxy)-3-methoxybenzoate (Compound (60))

To a solution of compound (D) (0.6 g, 1.19 mmol) in dichloromethane (12 ml), diisopropylethylamine (0.21 ml, 1.19 mmol) was added. The reaction was cooled at 0° C. and vanillic acid 4-(nitrooxy)butyl ester (0.34 g, 1.19 mmol) was added. The reaction was stirred at room temperature for hours. The solvent was evaporated under vacuum. The residue was purified by flash chromatography (Biotage System, SNAP Cartridge silica 50 g, eluent: gradient n-hexane/ethyl acetate 8/2 (75 ml), to n-hexane/ethyl acetate 2/8 during 525 ml, n-hexane/ethyl acetate 2/8 (225 ml)). The product (0.34 g) was obtained.

¹H-NMR: (DMSO), δ: 7.65 (1H, s), 7.64 (1H, d, J=7.9 Hz), 7.39 (1H, d, J=7.9 Hz), 7.31 (1H, d, J=10.1 Hz), 6.17 (1H, d, J=10.1 Hz), 5.93 (1H, s), 5.07 (1H, d, J=17.2 Hz), 4.91 (1H, d, J=17.2 Hz), 4.80 (1H, d, J=3.4 Hz), 4.60 (2H, t, J=5.5 Hz), 4.51 (2H, t, J=6.2 Hz), 4.32 (2H, t, J=5.0 Hz), 3.89 (3H, s), 2.75 (1H, t, J=12.2 Hz), 2.62-2.39 (3H, m), 2.30 (1H, dd, J=11.8, 3.7 Hz), 2.15-1.49 (15H, m), 1.38 (3H, s), 1.36 (1H, m), 1.03 (1H, m), 0.91 (3H, s).

### EXAMPLE 3

### Synthesis of (6S,9R,10S,11S,13S,16R,17R)-6,9-difluoro-11-hydroxy-17-(2-hydroxyacetyl)-10,13,16-trimethyl-3-oxo-6,7,8,9,10,11,12,13,14,15,16,17-dodecahydro-3H-cyclopenta[a]phenanthren-17-yl 4-nitroxybutanoate (compound (20))

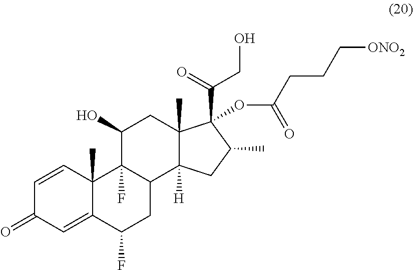

### F) (4′R,6S,9R,10S,11S,13S,16R)-2′-(3-bromopropyl)-6,9-difluoro-11-hydroxy-2′-methoxy-10,13,16-trimethyl-7,8,9,10,11,12,13,14,15,16-decahydrospiro[cyclopenta[a]phenanthrene-17,4′-[1,3]dioxane]-3,5′(6H)-dione

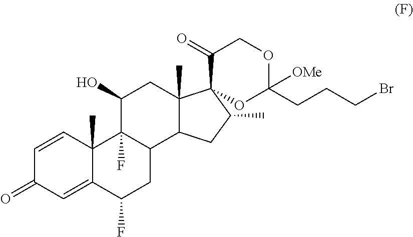

To a solution of flumethasone (0.9 g, 2.19 mmol) in toluene (15 ml) and N,N-dimethylformamide (2.1 ml), p-toluenesulfonic acid (cat) and trimethyl-4-bromo-orthobutyrate (0.76 ml, 4.38 mmol) were added. The reaction was stirred at room temperature for 72 hours. The mixture was poured in water (40 ml) and extracted with ethyl acetate (40×4 ml), the organic layers were dried over sodium sulfate and concentrated under reduced pressure. The residue was purified by flash chromatography, (Biotage System, SNAP Cartridge silica 100 g, eluent: gradient n-hexane/ethyl acetate 8/2 (145 ml), to n-hexane/ethyl acetate 3/7 during 870 ml, n-hexane/ethyl acetate 3/7 (725 ml)). The product (F) (0.89 g) was obtained.

### G) (8S,9S,10R,11S,13S,14S,17R)-11-hydroxy-17-(2-hydroxyacetyl)-10,13-dimethyl-3-oxo-6,7,8,9,10,11,12,13,14,15,16,17-dodecahydro-3H-cyclopenta[a]phenanthren-17-yl 4-bromobutanoate

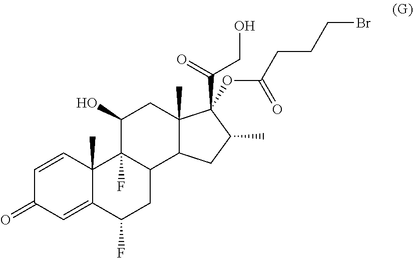

To a solution of compound (F) (0.88 g, 1.53 mmol) in methanol (27 ml), a 5% aqueous AcOH solution (5.5 ml) was added. The reaction was stirred a reflux for 7 hours. The mixture was concentrated under reduced pressure. The mixture was diluted with dichloromethane (30 ml), washed with saturated aqueous sodium carbonate (2×30 ml), water (2×30 ml), the organic layers were dried over sodium sulfate and concentrated under reduced pressure. The residue was purified by flash chromatography, (Biotage System, SNAP Cartridge silica 50 g, eluent: gradient acetone/dichlorometane 9/1 (75 ml), to acetone/dichlorometane 25/75 during 375 ml, acetone/dichlorometane 25/75 (375 ml)). The product (G) (0.74 g) was obtained.

### H) (6S,9R,10S,11S,13S,16R,17R)-6,9-difluoro-1′-hydroxy-17-(2-hydroxyacetyl)-10,13,16-trimethyl-3-oxo-6,7,8,9,10,11,12,13,14,15,16,17-dodecahydro-3H-cyclopenta[a]phenanthren-17-yl 4-nitroxybutanoate (compound (20))

To a solution of compound (G) (0.73 g, 1.31 mmol) in acetonitrile (32 ml), silver nitrate (0.67 g, 3.95 mmol) was added. The reaction was heated to 130° C. for 15 minutes under microwave irradiation. The resulting mixture was cooled, filtered and the solvent was removed under reduced pressure. The residue was diluted with dichloromethane (40 ml), washed with water (2×40 ml), the organic layer was dried over sodium sulfate and concentrated under reduced pressure. The crude was purified by flash chromatography, (Biotage System, SNAP Cartridge silica 50 g, eluent: gradient acetone/dichlorometane 9/1 (75 ml), to acetone/dichlorometane 25/75 during 375 ml, acetone/dichlorometane 25/75 (375 ml)). The product (0.57 g) was obtained.

¹H-NMR: (DMSO), δ: 7.25 (1H, d, J=10.1 Hz), 6.29 (1H, d, J=10.1 Hz), 6.11 (1H, s), 5.63 (1H, dq, J_{H-F}=48.8, J_{H-H}=10.8, 6.5 Hz), 5.48 (1H, d, J=3.6 Hz), 5.07 (1H, t, J=5.9 Hz), 4.51 (2H, t, J=6.4 Hz), 4.17 (1H, dd, J=17.2, 5.9 Hz), 4.16 (1H, bs), 4.05 (dd, J=17.2, 5.9, 1H), 3.28-3.16 (1H, m), 2.64-2.38 (3H, m), 2.25 (1H, m), 2.17-2.01 (2H, m), 1.99-1.72 (3H, m), 1.66 (1H, d, J=13.8 Hz), 1.49 (1H, m), 1.48 (3H, s), 1.23 (1H, m), 0.93 (3H, s), 0.84 (3H, d, J=6.9 Hz),

### EXAMPLE 4

### Synthesis of 4-(nitrooxy)butyl 4-((2-((6S,9R,10S,11S,13S,16R,17R)-6,9-difluoro-11-hydroxy-17-(4-nitroxybutanoyloxy)-10,13,16-trimethyl-3-oxo-6,7,8,9,10,11,12,13,14,15,16,17-dodecahydro-3H-cyclopenta[a]phenanthren-17-yl)-2-oxoethoxy)carbonyloxy)-3-methoxybenzoate (Compound (22))

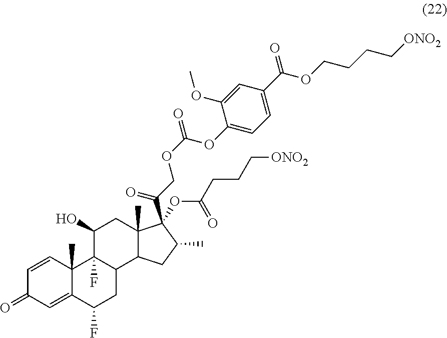

### I) (6S,9R,10S,11S,13S,16R,17R)-17-(2-(chlorocarbonyloxy)acetyl)-6,9-difluoro-11-hydroxy-10,13,16-trimethyl-3-oxo-6,7,8,9,10,11,12,13,14,15,16,17-dodecahydro-3H-cyclopenta[a]phenanthren-17-yl 4-nitroxybutanoate

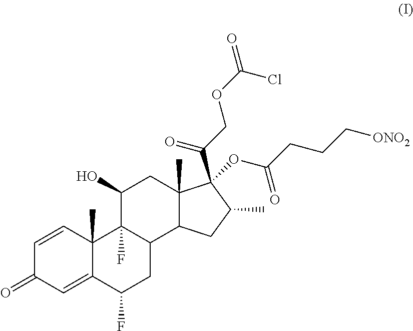

To a solution of compound (20) (0.35 g, 0.64 mmol) in tetrahydrofurane (3.1 ml), cooled at 0° C. and under N₂, a 20% toluene solution of phosgene (2.03 ml, 3.87 mmol) was added. The reaction was stirred at 0° C. for 1 hour and at room temperature for 22 hours. The excess of phosgene was removed by heating at 40° C. for 45 minutes. The solvent was evaporated under vacuum. The crude product (I) (0.38 g) was used in the next step without any purification.

### L) 4-(nitrooxy)butyl 4-((2-((6S,9R,10S,11S,13S,16R,17R)-6,9-difluoro-11-hydroxy-17-(4-nitroxybutanoyloxy)-10,13,16-trimethyl-3-oxo-6,7,8,9,10,11,12,13,14,15,16,17-dodecahydro-3H-cyclopenta[a]phenanthren-17-yl)-2-oxoethoxy)carbonyloxy)-3-methoxybenzoate Compound (22)

To a solution of compound (I) (0.38 g, 0.63 mmol) in dichloromethane (7 ml), diisopropylethylamine (0.12 ml, 0.69 mmol) was added. The reaction was cooled at 0° C. and vanillic acid 4-(nitrooxy)butyl ester (0.19 g, 0.69 mmol) was added. The reaction was stirred at room temperature for hours. The solvent was evaporated under vacuum. The residue was purified by flash chromatography (Biotage System, SNAP Cartridge silica 50 g, eluent: gradient n-hexane/ethyl acetate 9/1 (75 ml), to n-hexane/ethyl acetate 2/8 during 675 ml, n-hexane/ethyl acetate 2/8 (375 ml)).

The product (0.38 g) was obtained.

¹H-NMR: (DMSO), δ 7.65 (1H, s), 7.64 (1H, d, J=8.1 Hz), 7.39 (1H, d, J=7.1 Hz), 7.25 (1H, d, J=10.2 Hz), 6.29 (1H, d, J=10.2 Hz), 6.11 (1H, s), 5.64 (1H, dq, =49.0, J_{H-H}=10.6, 6.6 Hz), 5.49 (1H, d, J=3.1 Hz), 5.16 (1H, d, J=16.5 Hz), 4.92 (1H, d, J=16.5 Hz), 4.60 (2H, t, J=5.5 Hz), 4.52 (2H, t, J=6.1 Hz), 4.34 (2H, t, J=5.1 Hz), 4.19 (1H, m), 3.89 (3H, s), 2.55 (2H, t, J=7.4 Hz), 2.51 (1H, m), 2.25 (1H, m), 2.19-2.02 (2H, m), 1.98-1.74 (8H, m), 1.66 (1H, d, J=13.8 Hz), 1.50 (1H, m), 1.48 (3H, s), 1.23 (1H, m), 0.99 (3H, s), 0.89 (3H, d, J=6.7 Hz).

1. A compound of formula (I) and pharmaceutically acceptable salts or stereoisomers thereof 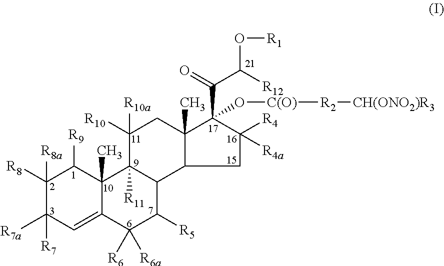
wherein:
R₁ is —H or R₁ is selected from
(A) —R¹—CH(NHR²)—C(O)—O—Y
(B) —R¹—CH(COOH)NH—C(O)—Y
(C) —R¹—CH(COOH)—O—C(O)—Y
(D) —C(O)CH(R³)—NH—C(O)—Y
(E) —C(O)CH₂—CH(R⁴)—NH—C(O)—Y
(F) —(Z)—Y
(G) 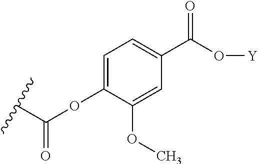
(H) 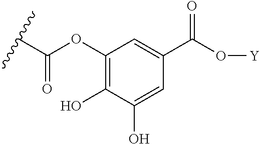
(I) 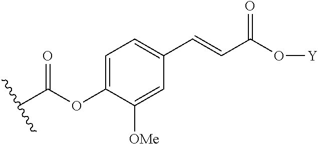
wherein:
R¹ is selected from
R^{1a}) 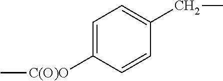
—C(O)—S—CH₂—, —C(O)O—CH(CH₃)—, —C(O)O—CH₂—;
R^{1b})
—C(O)—CH₂—, —C(O)—(CH₂)₂—;
R² is —H or —C(O)CH₃;
R³ is —H, —CH₃, isopropyl, isobutyl, sec-butyl, methylthio-(CH₂)₂—, benzyl;
R⁴ is —H, —CH₃, isopropyl, isobutyl, sec-butyl, methylthio-(CH₂)₂—, benzyl;
Z is —C(O) or —C(O)—X″, wherein X″ is O, S or NR₁₁ wherein R₁₁ is H or a C₁-C₄ alkyl;
R₂ is a straight or branched C₁-C₁₀ alkylene;
R₃ is H or a straight or branched C₁-C₄ alkyl;
R₄ is —H, —CH₃;
R_{4A} is —H,
or R₄ and R_{4A} taken together are ═CH₂;
R₅ is —H, Cl;
R₆ is —H, Cl, F, CH₃;
Rₑₐ is —H,
or R₆ and R₅ taken together are a double bond;
R₇ₐ is H,
or R₇ and R_{7A} taken together are ═O;
R₈ is H, Cl,
or R₇ and R₈ taken together are the group of formula (V) 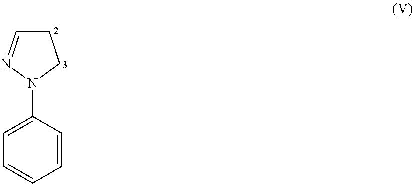
R₈ₐ is H,
R₉ is —H,
or R₈ₐ and R₉ taken together are a double bond
R₁₀ is —OH,
R₁₀ₐ is H,
or R₁₀ and R₁₀ₐ taken together are ═O;
R₁₁ is —H, —Cl, —F;
R₁₂ is —H, CH₃;
wherein R₄, R₄ₐ, R₅, R₆, R₆ₐ, R₇, R₇ₐ, R₈, R₈ₐ, R₉, R₁₀, R₁₀ₐ can be linked to the corresponding carbon atoms of the steroidal structure in position α or β;
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
wherein
R₁₃ is a straight or branched C₁-C₁₀ alkylene;
R₁₄ is H or a straight or branched C₁-C₄ alkyl;
R₁₅ and R₁₆ are H or a straight or branched C₁-C₁₀ alkylene;
o and r are integers from 1 to 6;
p and s are integers from 1 to 6;
q is an integer from 0 to 6;
t is an integer from 0 to 6;
X is O, S or NR₁₇ wherein R₁₇ is H or a C₁-C₄ alkyl;
excluding the following structures of formula (I):

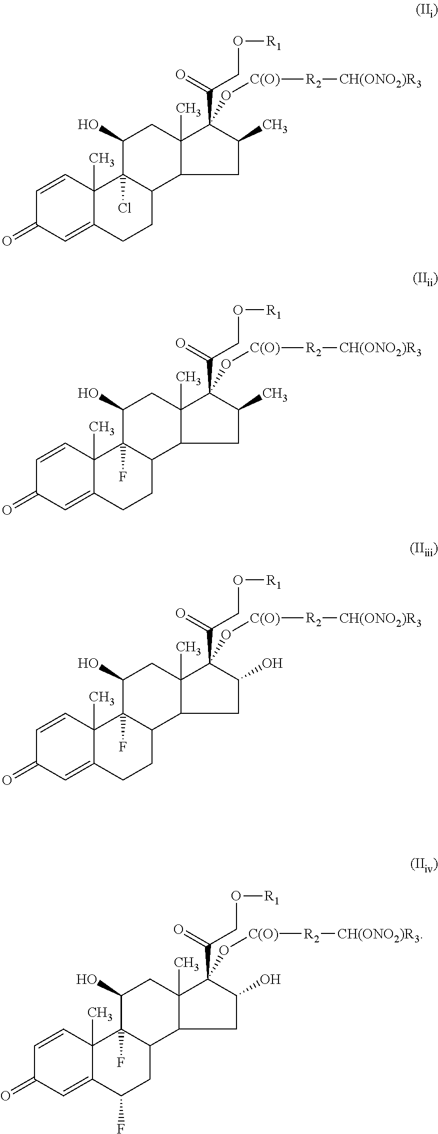

2. A compound according to claim 1 wherein R₁ is —H.

3. A compound according to claim 1 wherein
R₁ is
(F) —(Z)—Y
wherein:
Z is —C(O) or —C(O)—X″, wherein X″ is O, S or NR¹¹ wherein R₁₁ is H or a C₁-C₄ alkyl;
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
—[(CH₂)ₒ—X]ₚ—[(CH₂)ᵣ—X]ₛ—(CH₂)_{q}—CH(ONO₂)—(CR₁₅R₁₆)ₜ—CH(ONO₂)R₁₄
wherein
R₁₃ is a straight or branched C₁-C₁₀ alkylene,
R₁₄ is H or —CH₃,
R₁₅ and R₁₆ at each occurrence are independently H or —CH₃,
o and r are integers from 1 to 4,
p and s are integers from 1 to 4,
q is an integer from 0 to 4,
t is 0 or 1
X is 0.

4. A compound according to claim 1
wherein
R₁ is
(G) 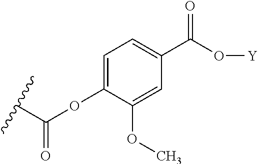
(H) 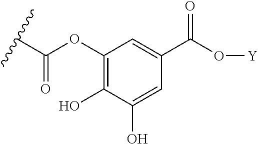
(I) 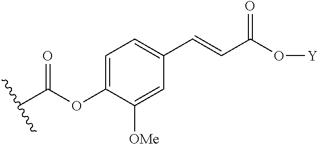
wherein:
Y is selected from
—R₁₃—CH(ONO₂)R₁₄
—R₁₃—CH(ONO₂)—(CR₁₅R₁₆)ₜ—CH(ONO₂)R₁₄
—[(CH₂)ₒ—X]ₚ—[(CH₂)ᵣ—X]ₛ—(CH₂)_{q}—(CR₁₅R₁₆)ₜ—CH(ONO₂)R₁₄
wherein
R₁₃ is a straight or branched C₁-C₁₀ alkylene,
R₁₄ is H or —CH₃,
R₁₅ and R₁₆ at each occurrence are independently H or —CH₃,
o and r are integers from 1 to 4,
p and s are integers from 1 to 4,
q is an integer from 0 to 4,
t is 0 or 1,
X is 0.

5. A compound according to claim 1
wherein
R₁ is
(A) —R¹—CH(NHR²)—C(O)—O—Y
(B) —R¹—CH(COOH)NH—C(O)—Y
(C) —R¹—CH(COOH)—O—C(O)—Y
(D) —C(O)CH(R³)—NH—C(O)—Y
(E) —C(O)CH₂—CH(R⁴)—NH—C(O)—Y
wherein:
Y is selected from
—R₁₃—CH(ONO₂)R₁₄
—R₁₃—CH(ONO₂)—(CR₁₅R₁₆)ₜ—CH(ONO₂)R₁₄
wherein
R₁₃ is a straight or branched C₁-C₁₀ alkylene,
R₁₄ is H or —CH₃,
R₁₅ and R₁₆ at each occurrence are independently H or —CH₃,
t is ° or 1.

6. A compound according to claim 5 wherein
R¹ of R^{1a}) is 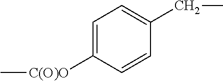
or
R¹ of R^{1b}) is —C(O)—CH₂—,
R³ is H or —CH₃,
R⁴ is —H or —CH₃.

7. A compound according to claim 1 selected from the followings

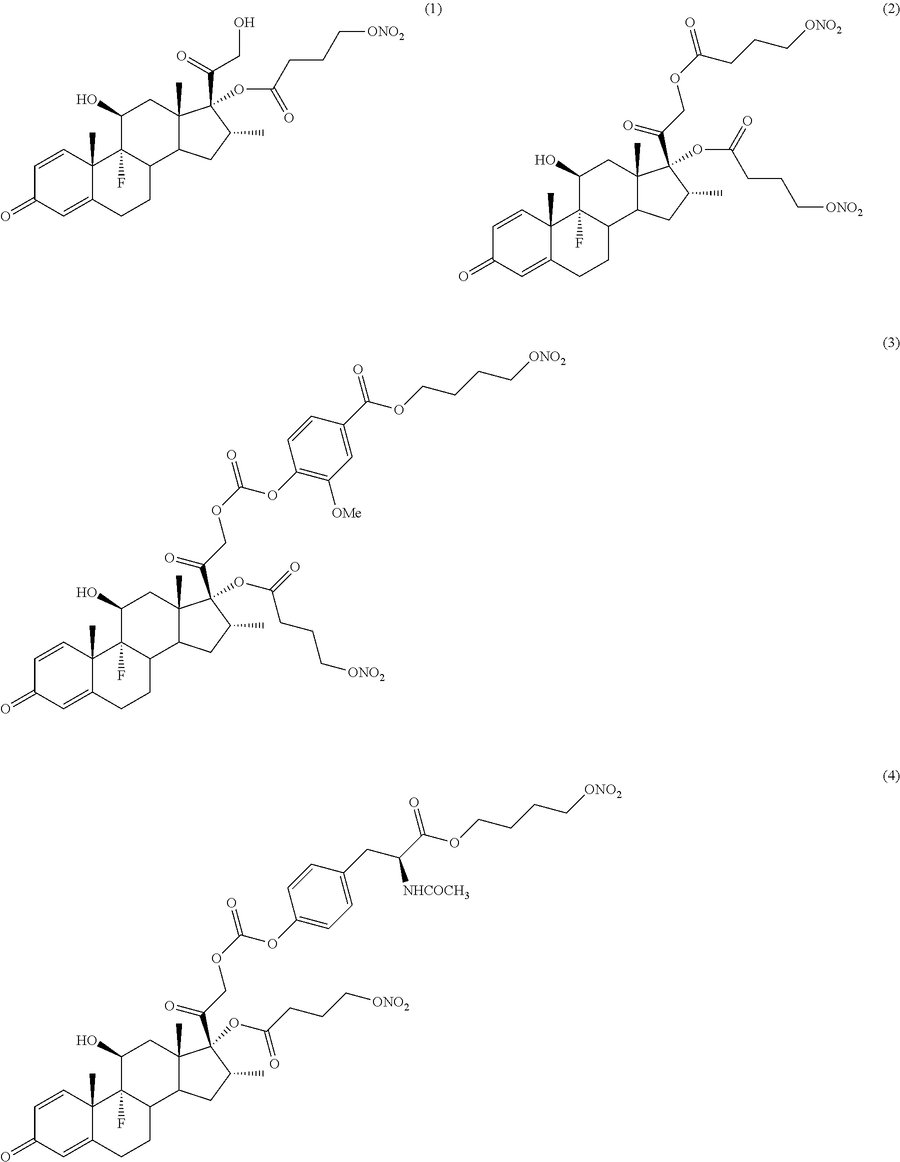

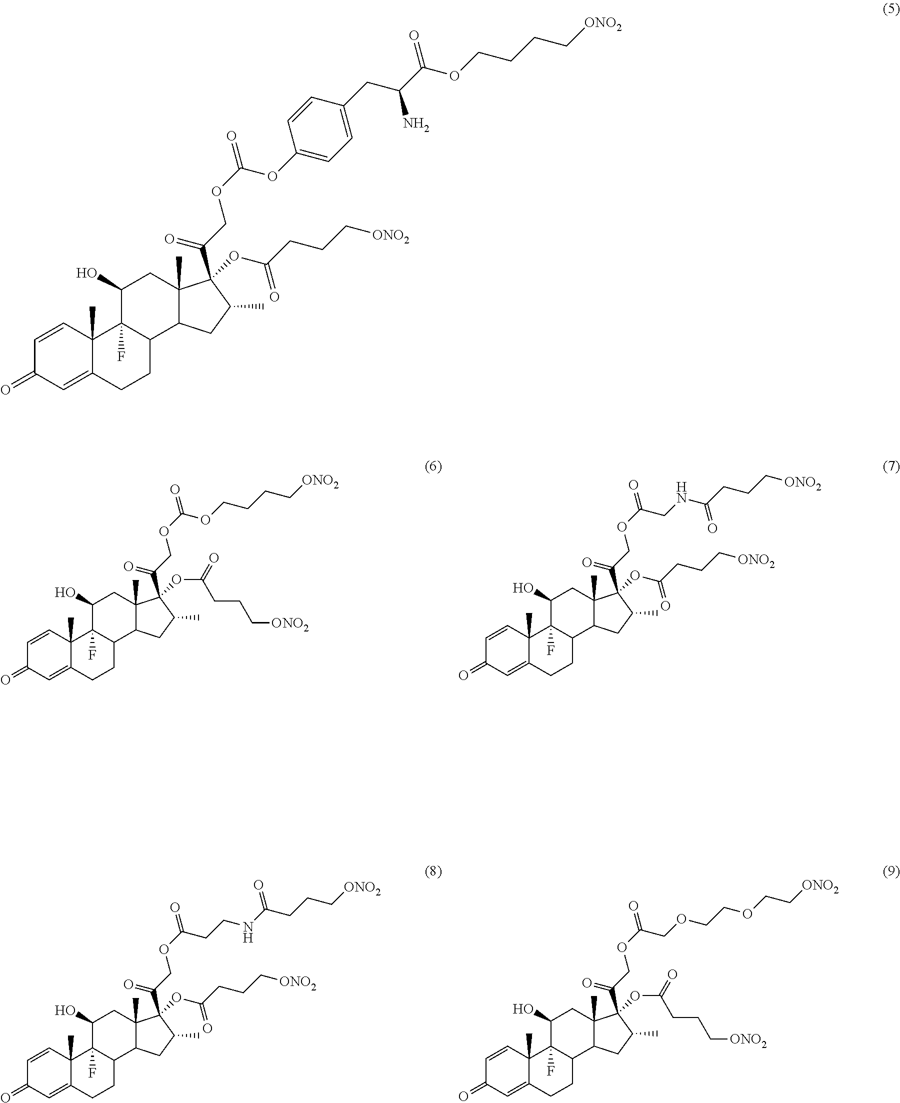

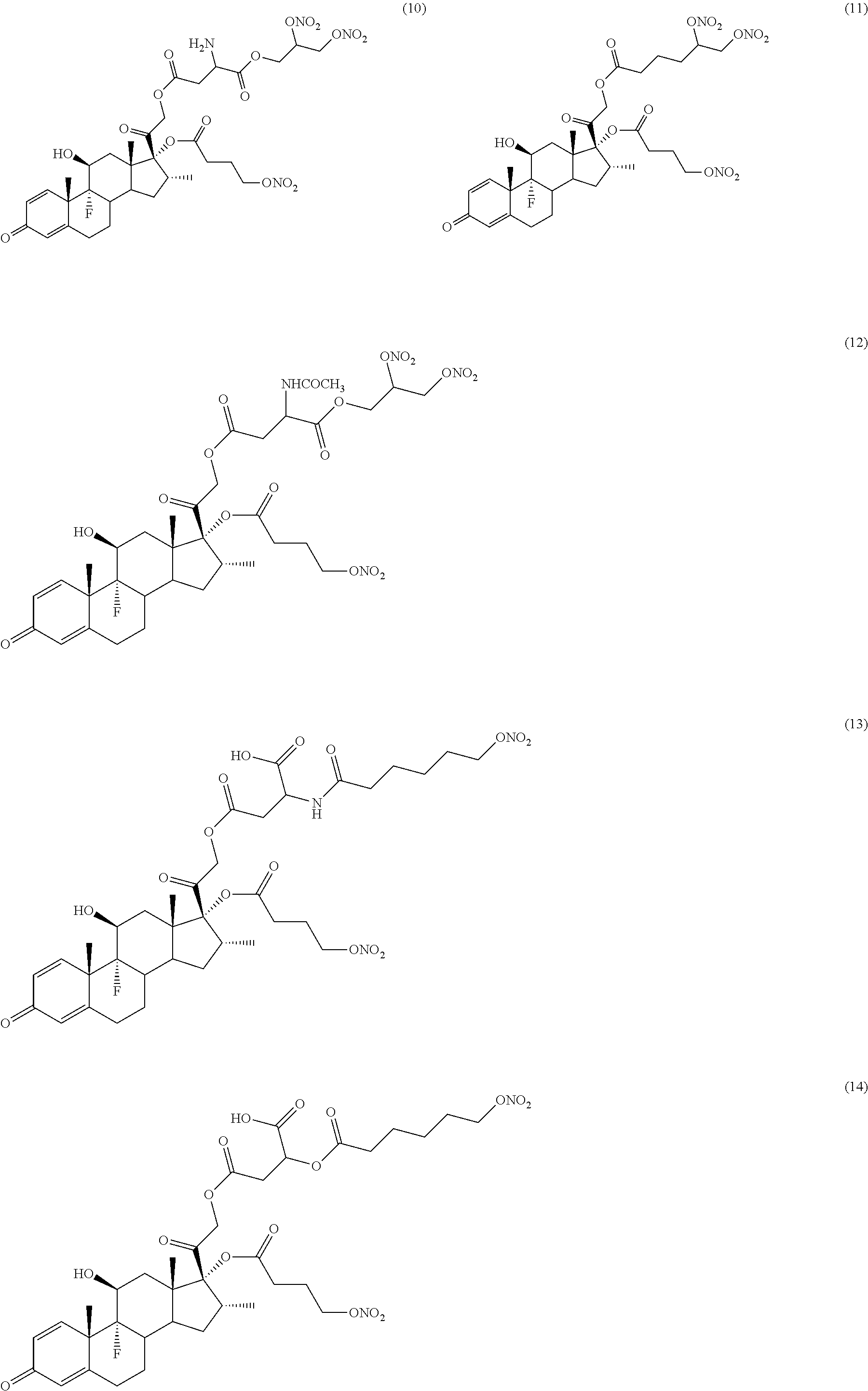

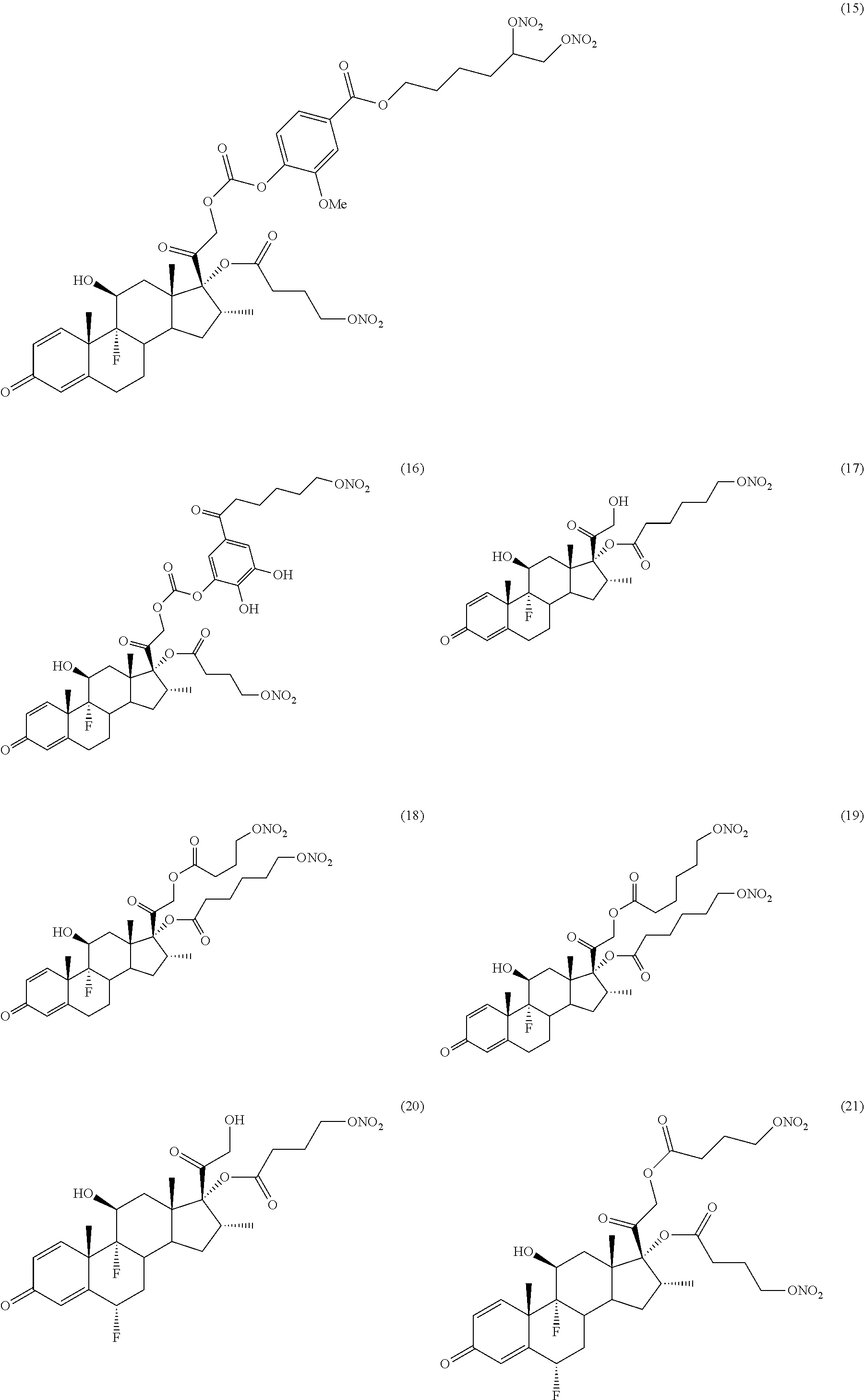

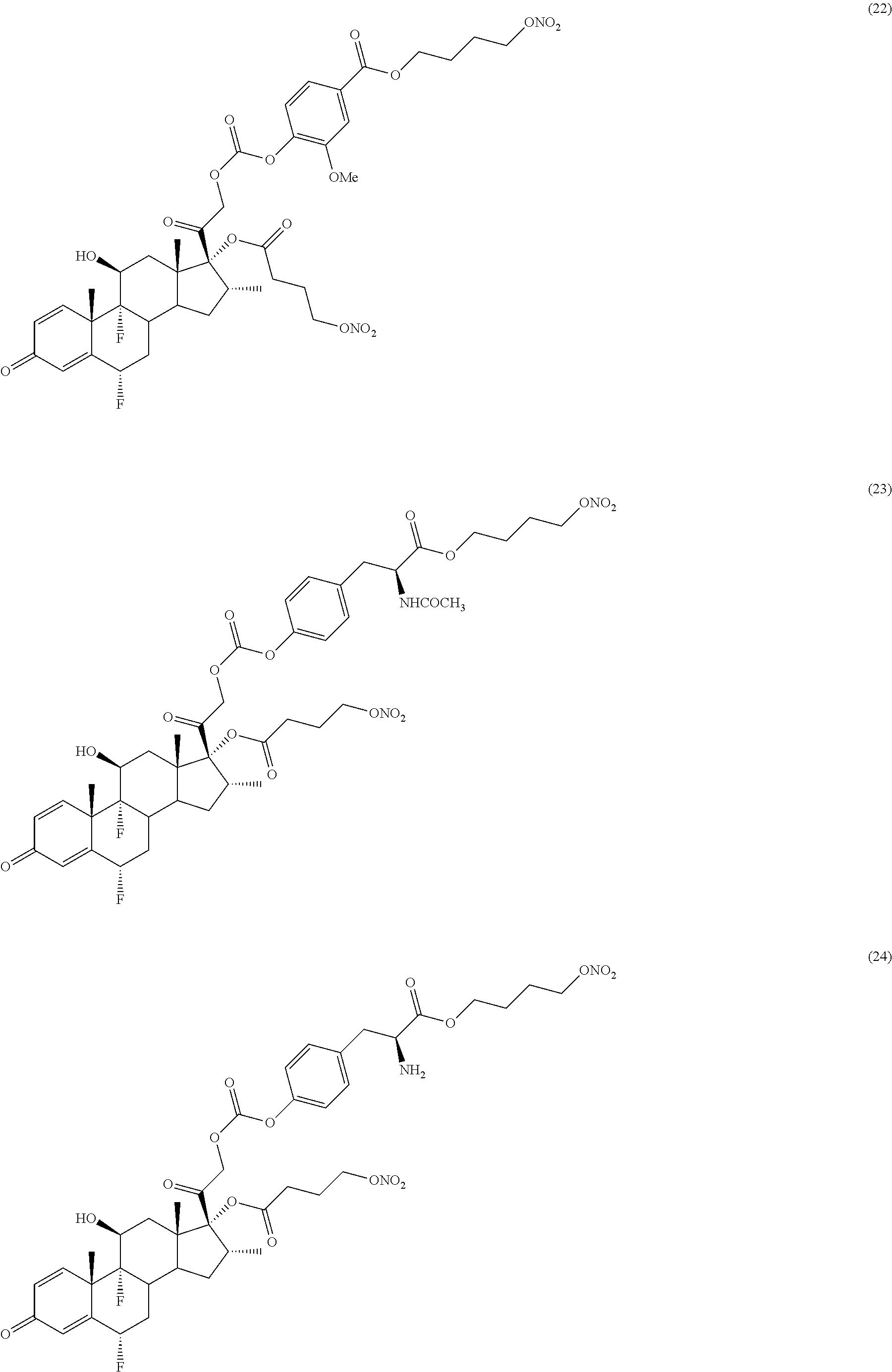

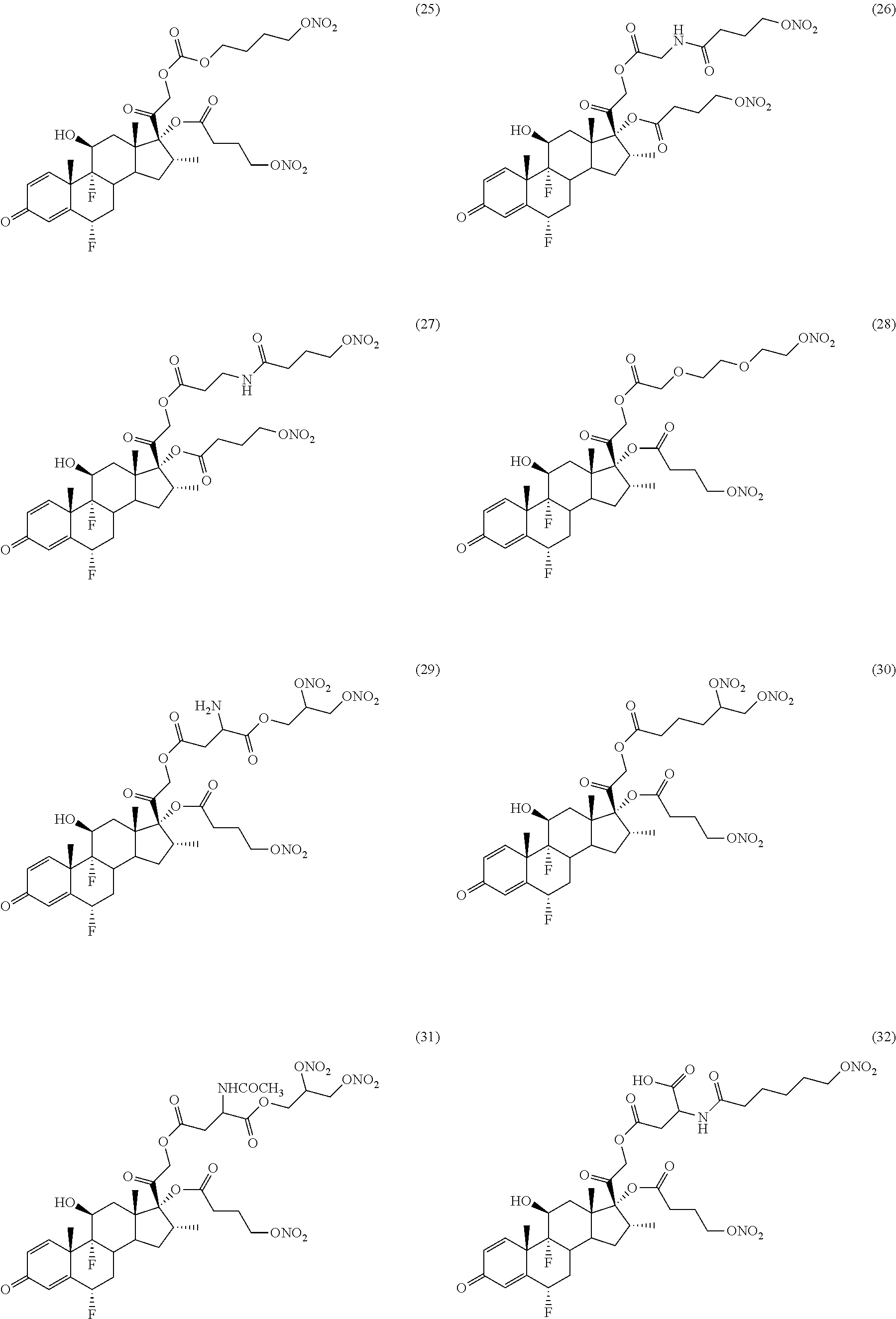

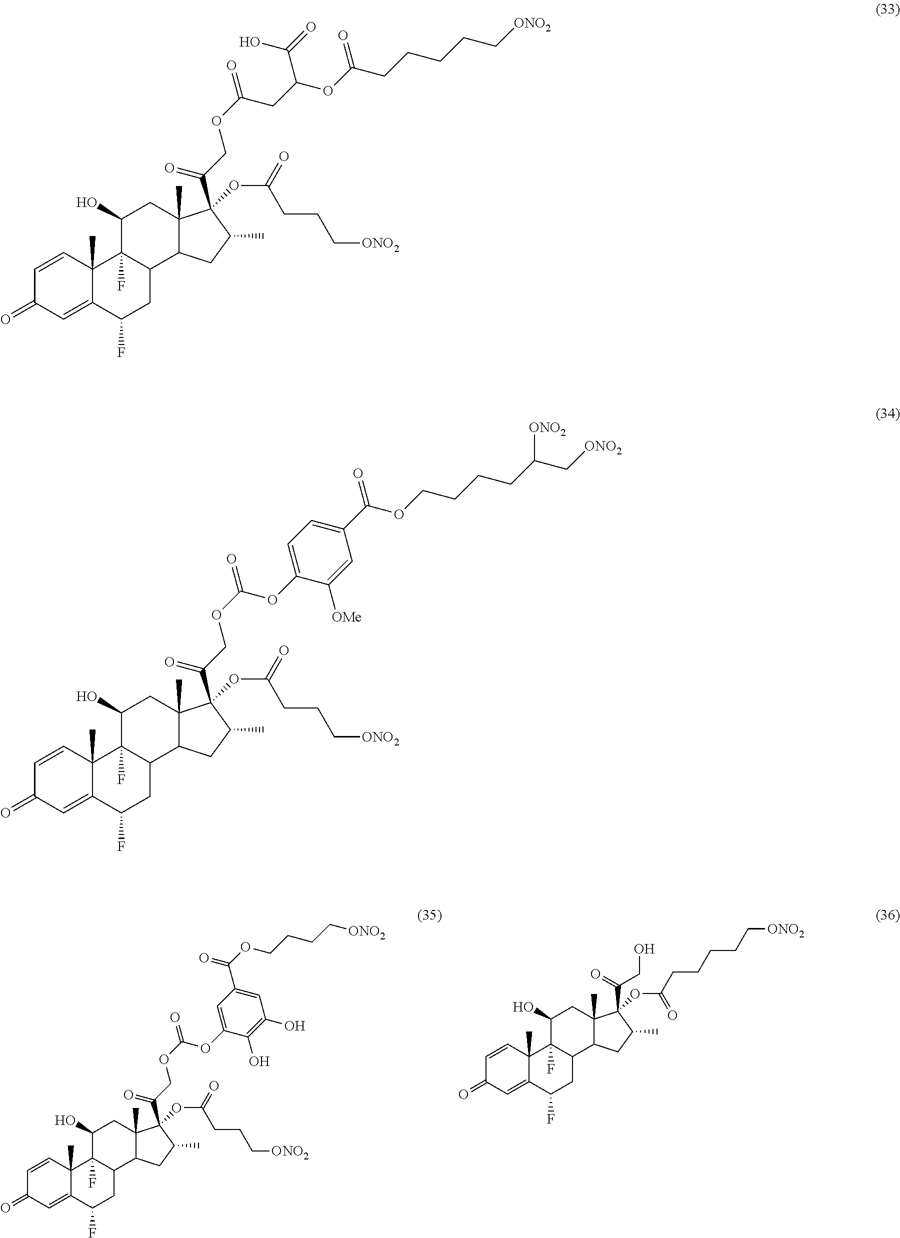

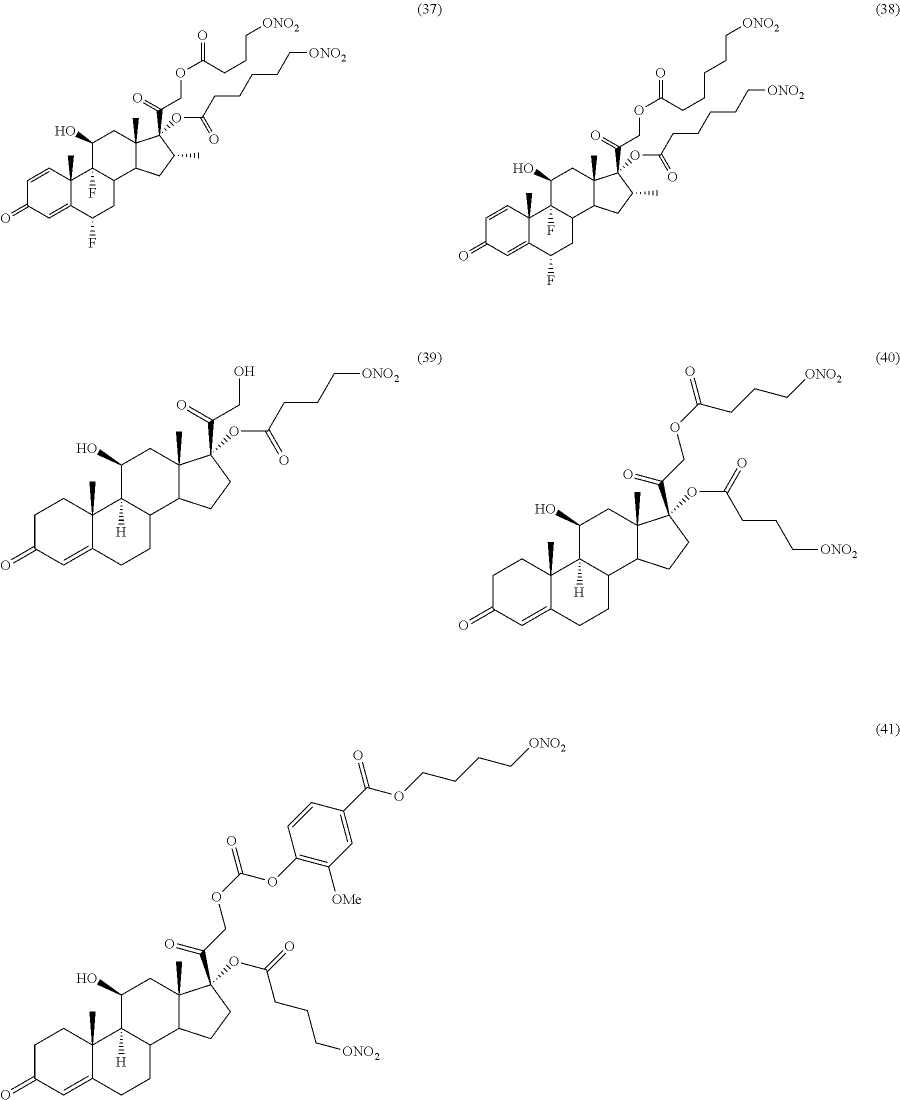

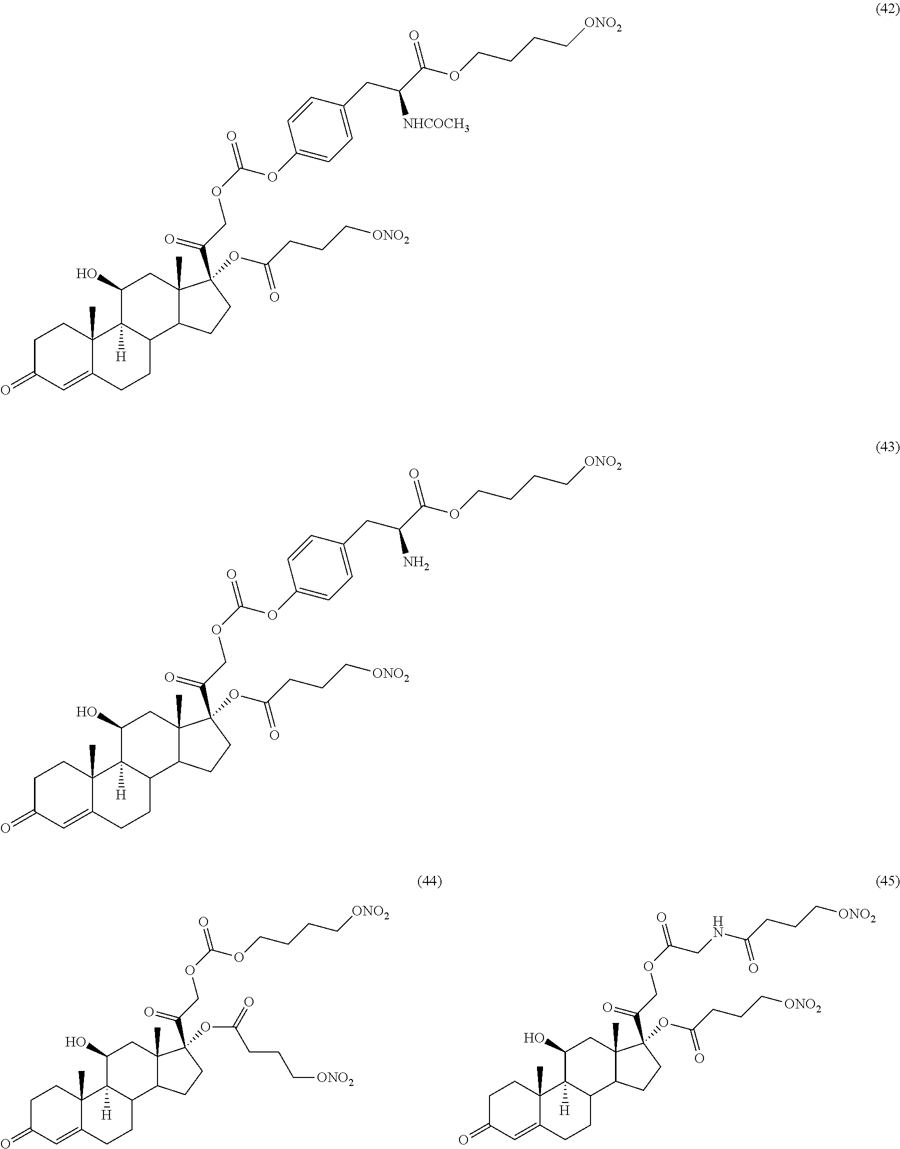

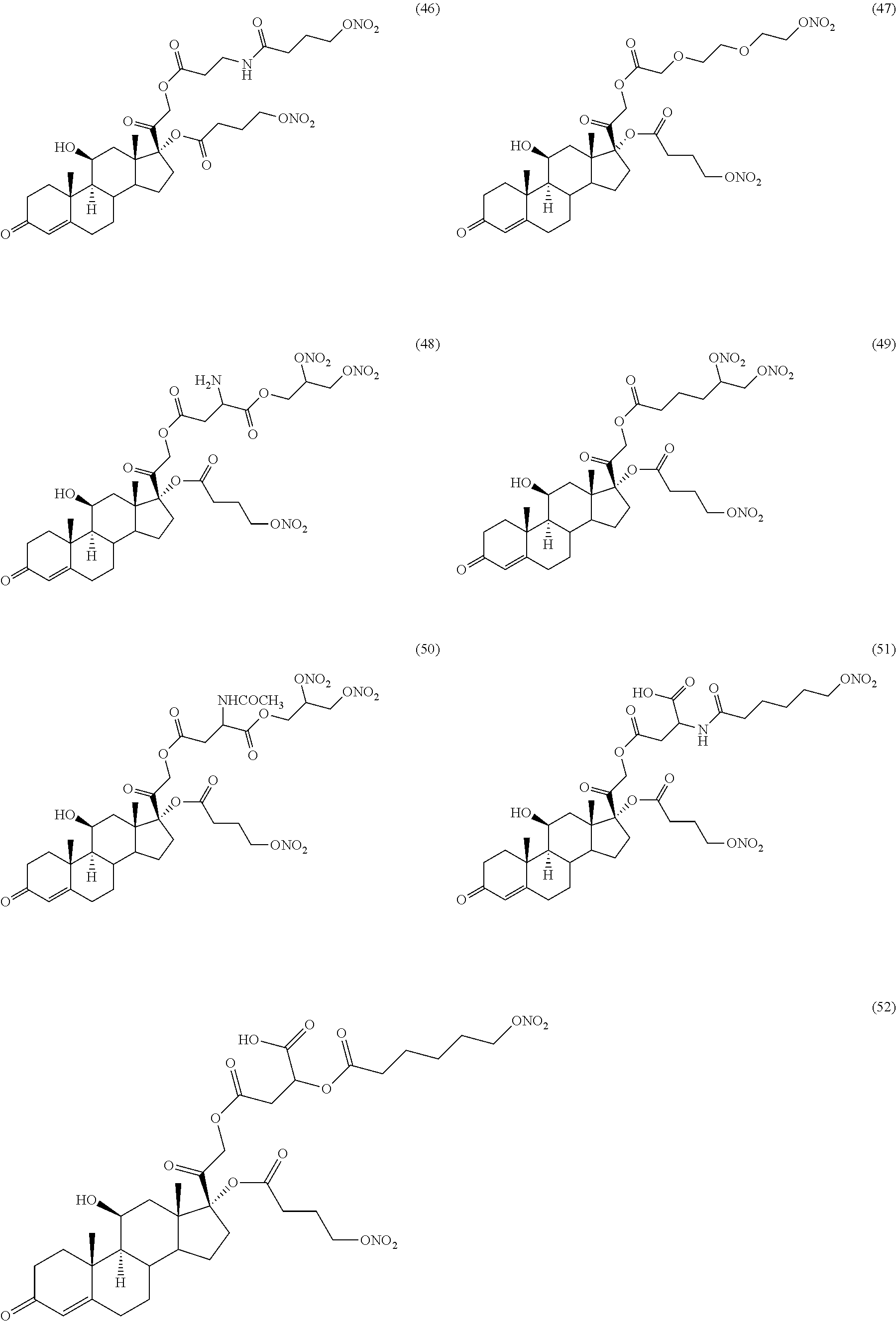

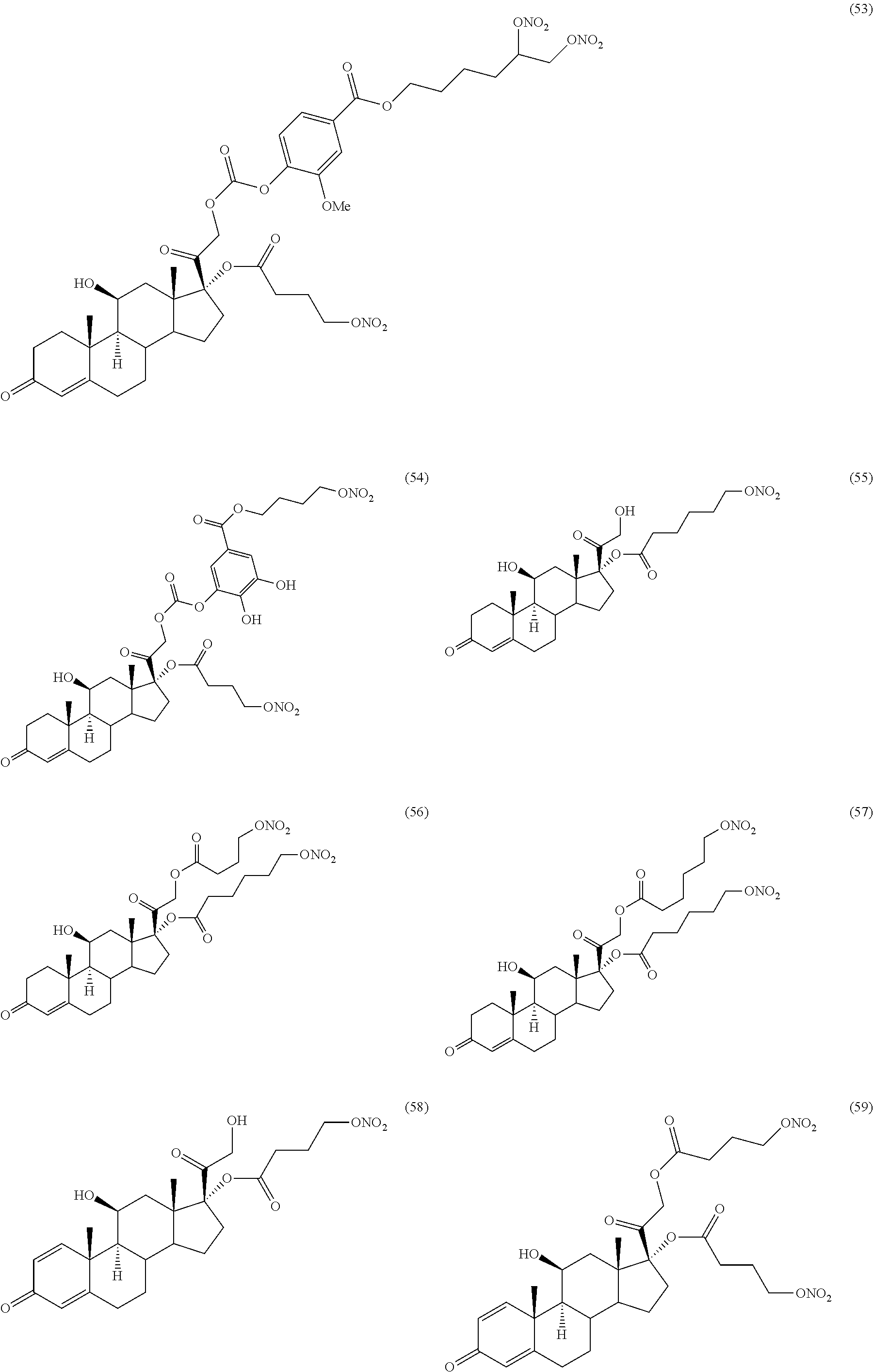

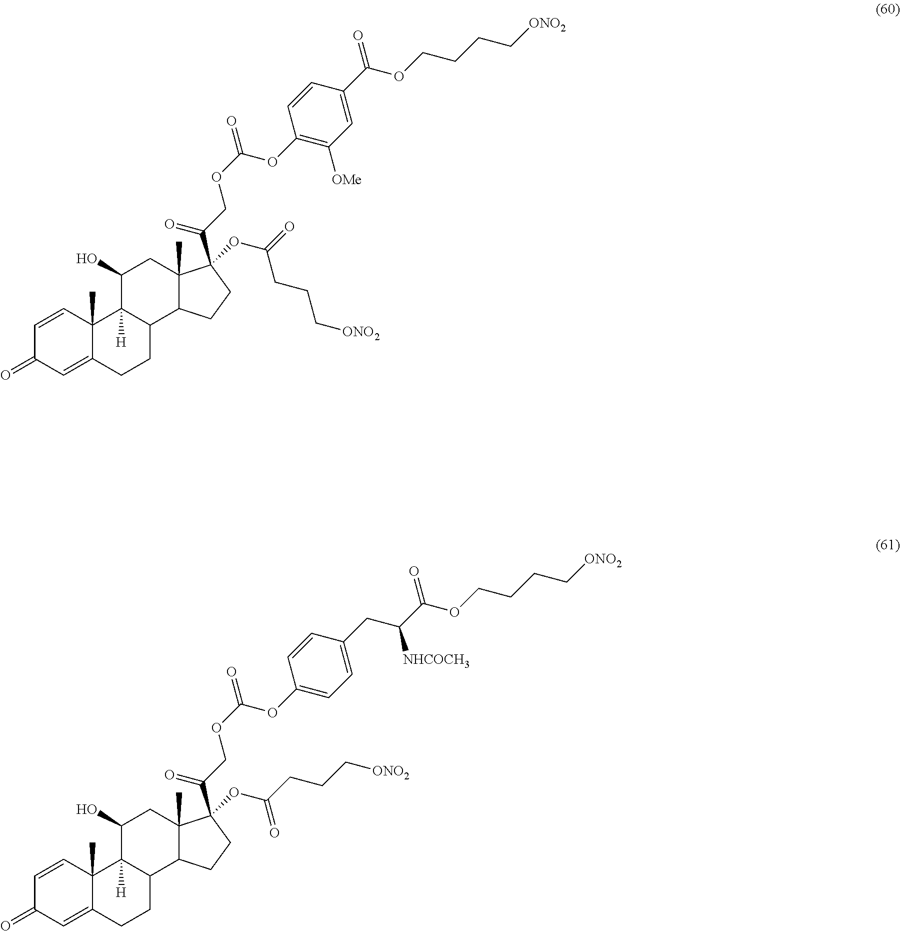

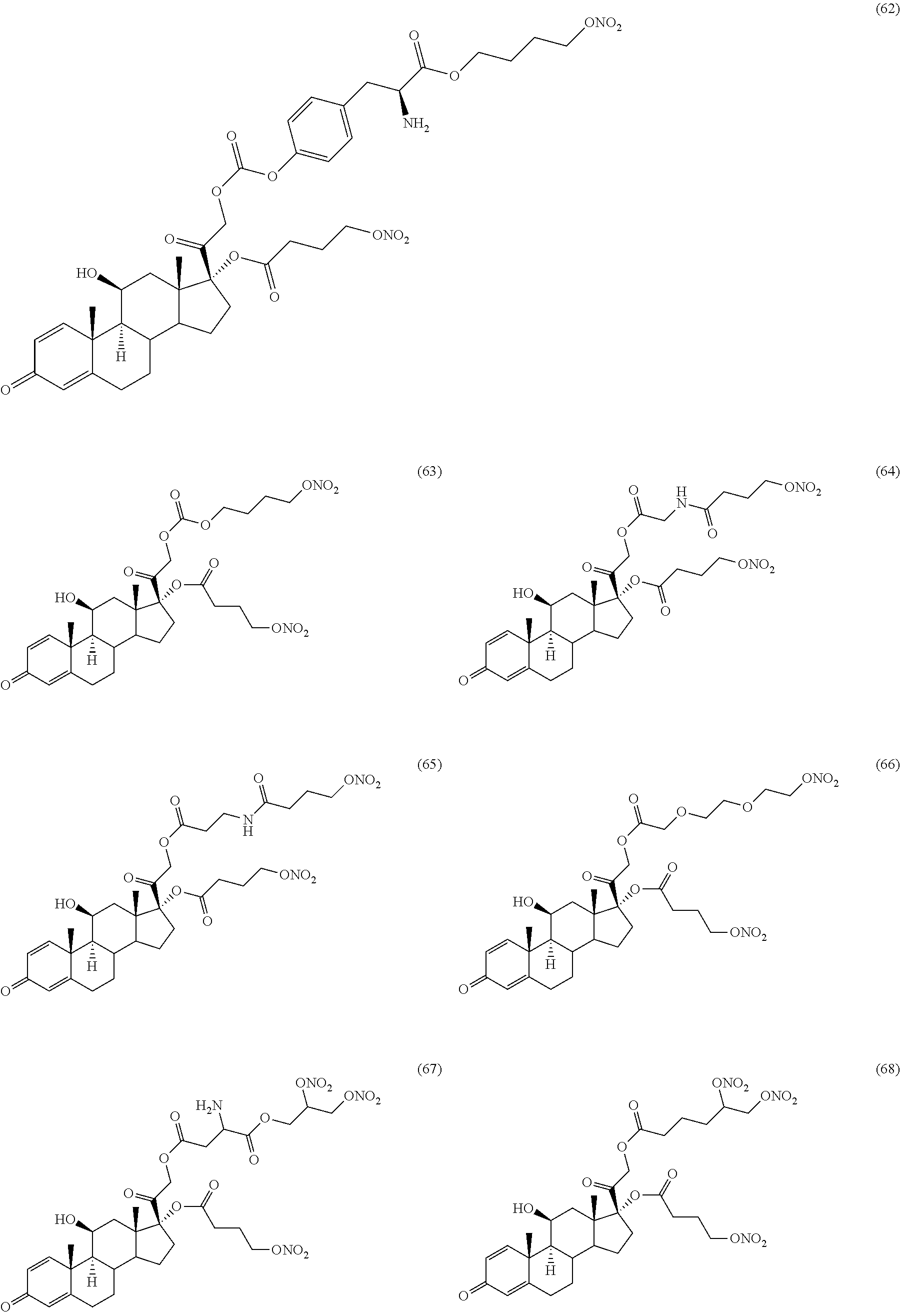

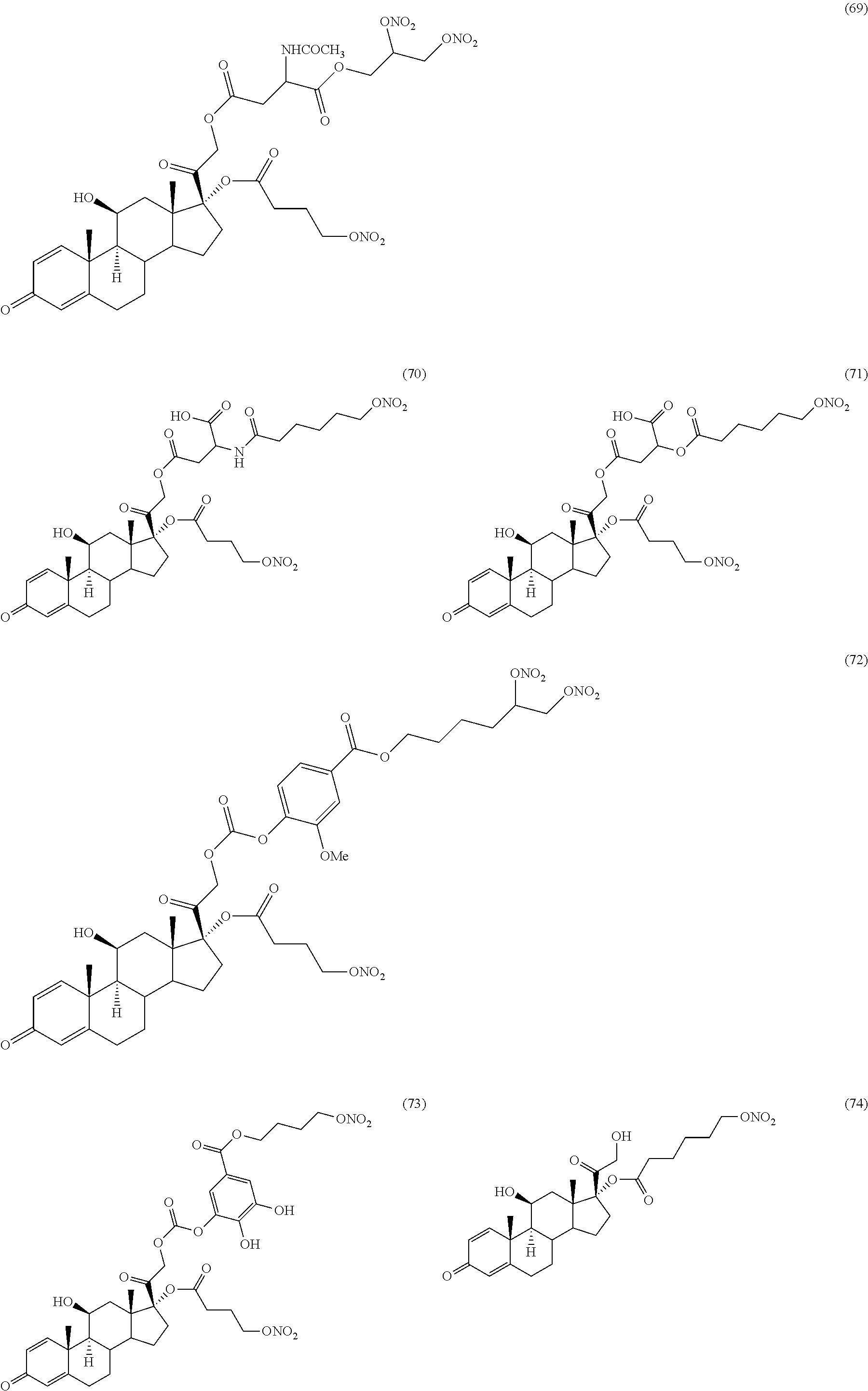

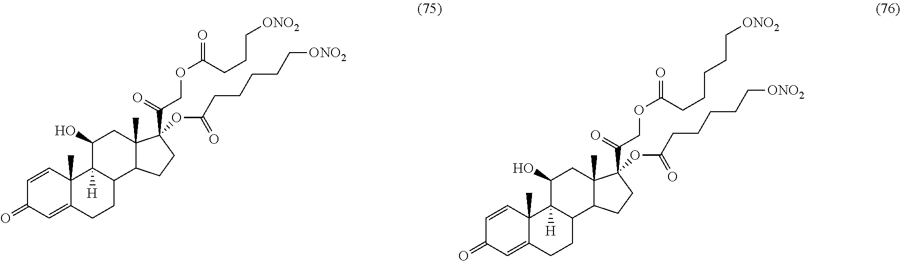

8. A compound according to claim 1 for use as medicament.

9. A compound according to claim 1 for use in the treatment of inflammatory diseases.

10. A compound according to claim 1 for use in the treatment of rheumatic diseases, renal and bronchial pathologies, ocular and dermatological diseases, autoimmune diseases, tumoral processes, also in combination with chemotherapeutic and/or radiotherapeutic treatments, in neurodegenerative diseases, for example in spinal lesions from trauma and in the post-transplant therapy.

11. Use of a compound according to claim 1 for the preparation of medicaments for the treatment of inflammatory diseases.

12. Use of a compound according to claim 1 for the preparation of medicaments for the treatment of rheumatic diseases, renal and bronchial pathologies, ocular and dermatological diseases, autoimmune diseases, tumoral processes, also in combination with chemotherapeutic and/or radiotherapeutic treatments, in neurodegenerative diseases, for example in spinal lesions from trauma and in the post-transplant therapy.

13. A pharmaceutical composition comprising a pharmaceutically effective amount of at least a compound according to claim 1, and a pharmaceutical acceptable carrier.
